(19) United States
(12) Patent Application Publication
(10) Pub. No.: US 2009/0181098 A1
(43) Pub. Date: Jul. 16, 2009

(54) Hmg-Co-a Reductase Inhibitor Enhancement of Bone and Cartilage

(75) Inventors: Ian R Garrett, Helotes, TX (US); Gloria Gutierrez, San Antonio, TX (US); Gianni Rossini, San Antonio, TX (US); Samuel P. Sawan, Tyngsboro, MA (US); Gregory R. Mundy, San Antonio, TX (US)

Correspondence Address: MARTIN D. MOYNIHAN d/b/a PRTSI, INC., P.O. BOX 16446, ARLINGTON, VA, 22215, US

(73) Assignee: OsteoScreen IP, LLC, San Antonio, TX (US)

(21) Appl. No.: 12/224,813

(22) PCT Filed: Mar. 6, 2007
(86) PCT No.: PCT/US07/05684

§ 371 Date: Mar. 13, 2009

Related U.S. Application Data
(60) Provisional application No. 60/779,434, filed on Mar. 7, 2006.
(60) Provisional application No. 60/812,987, filed on Jun. 13, 2006.
(60) Provisional application No. 60/831,938, filed on Jul. 20, 2006.

(51) Int. Cl. A61K 9/14; A61P 19/08

(52) U.S. Cl. 424/489

(57) ABSTRACT

Methods of enhancing skeletal framework tissue are provided by treating a site requiring enhancement with an HMG-CoA reductase inhibitor at a dosage and for a duration that enhances the tissue while avoiding excess of the inhibitor and degradation of the enhancement.

### TECHNICAL FIELD

The field of this invention is the enhancement of bone and cartilage.

### BACKGROUND

The vertebrate skeleton is made up of bone and cartilage. Other bone containing body parts are teeth. The formation of bone and cartilage plays a major role in the maintenance and repair of vertebrates. Of particular interest are primates, more particularly humans. The numerous problems associated with the deterioration of bone and cartilage, the loss of bone as in osteoporosis and tooth extractions and breaking and compaction of bone, tearing and wear of cartilage, etc. are common events requiring a substantial proportion of the total medical activity. These various detriments can result in severely damaging the host, the inability to move where traction and casts are involved, the pain and suffering endured during the recovery, the inability to work, and the requirement for supporting devices. These procedures and events add a substantial cost and burden to the public and to medical support groups.

Great progress has been made in the use of pins and prostheses in repairing many bone injuries. However, the use of non-anatomic materials, such as metals and plastics frequently results in weak bonding between the non-anatomic materials and the native tissue. Various techniques have been used to improve the bonding of the prosthesis to the bone, using osteoconductive materials, such as hydroxyapatite, demineralized bone, calcium phosphates, etc., with varying degrees of success.

Bone fractures have always been problematic for mankind and treatment has remained essentially unchanged for centuries. AAOS statistics indicate approximately 6.8 million fractures occur each year in the US and over the course of a lifetime, each person will, on average, experience two fractures. More than 900,000 hospitalizations result each year from fractures. Normal fracture healing is a complex, multi-step process involving cellular events influenced and regulated by local and systemic factors. However, the most common biological failure in fracture healing involves an improperly formed callus within the first weeks after the fracture. In the case of fractures, one is interested in minimizing the time it takes to allow the repaired bone to be weight bearing. Where fusion of bones is dictated, a strong bond that is quickly formed can substantially reduce the incapacity of the patient. In most situations one is interested in the rapidity with which the new cartilage or bone is formed, the strength of the new structure, the absence of side effects from the treatment, minimizing pain and inflammation, and providing adequate restoration of the cartilage or bone.

It is known that members of the bone morphogenetic protein (“BMP”) family activate osteoblasts and chondrocytes, both of which have receptors for the members of the BMP family. It is also known that statins induce BMP formation. See, for example, U.S. Pat. Nos. 6,022,887 and 6,080,779, as well as U.S. Pat. Nos. 7,041,309 and 7,108,862, all of whose disclosures are specifically incorporated herein by reference as if set forth herein as to their disclosures of the use of statins in producing bone and cartilage. The methods described employ oral administration or involve an incision to open the anatomic site to direct application of the statin formulation. While the references refer to various other methods of administration, these are not specifically exemplified, nor are they shown to have improved results.

There is a need for effective modes of administration of therapeutic compositions that provide for bone and cartilage enhancement within shortened periods of time to allow unsupported use of the skeletal or dental part with minimal side effects and ease of administration as to dose and regimen. While a wide variety of methods of application of the statins have been taught in the patent literature, basically a litany of all methods known, the methods for the actual testing of the statins for their inducing bone formation have been very limited, possibly suggesting that, in fact, other methods were not promising.

Statins are known to result in a wide variety of effects, both therapeutic and deleterious to the host. As in so many cases, the desirable aspects are accepted in light of the therapeutic results, where in may cases the deleterious effects can be minimized by further administration of other drugs. There is, therefore, a substantial interest in being able to provide for therapeutic dosages of HMG Co-A reductase inhibitors, such as statins while minimizing side effects and avoiding ineffective levels of the drug.

### RELEVANT LITERATURE

U.S. Pat. Nos. 6,022,887 and 6,080,779, as well as U.S. Patent application nos. 2003/0232065 and 2004/0006125, and references cited therein, describe the use of statins for the promotion of bone and cartilage. Skoglund and Aspenberg, 52^{nd} Annual Meeting of the Orthopaedic Research Society, 2006/1667 in a poster describe using a minipump for the administration of statins to enhance bone formation.

Studies with rats have shown that the occurrence of BMP, OP and their receptors in bone cells and fractures in rats is restricted in the time of occurrence and their duration. Short time expression is sufficient for in vivo osteochondral differentiation of cells and the 5-6 days dosing is optimal. (Noel, et al. 2004 Stem Cells 22, 74-85) Expression of BMP and OP 1 and their appropriate receptors in a fracture is strongly expressed at 1, 2 weeks, decreased at 4 weeks and not present at week 8 in rats. (Orishi, et al. 1998 Bone 22, 605-12) Further support is found in that BMP expression is disappearing at 4 weeks and gone at 8 weeks in a rat healing mandible. (Spector, et al. 2001 Plast Reconstr Surg 107, 124-34) In a study of BMP receptor expression at weekly intervals in a rabbit model of distraction osteogenesis, BMP receptors are strongly upregulated at week 2, but downregulated by week 4-5. (Hamdy, et al. 2003 Bobe 33, 248-55) Also involved in bone healing is the expression of the BMP activity inhibitor Noggin. It was found that Noggin was strongly expressed after Day 5 in mouse fracture callus. Injection of BMP in a young mouse at fracture Day 0, Day 4 and Day 8 days and then assessed at Day 22 showed that the early administration of BMP were most effective at Day 0 and 4. (Murnaghan, et al. 2005 J Orthop Res 23, 625-31) When a sheep critical size defect is treated with adenoviral vectors encoding BMP2, the healing of the defect was retarded at 8 weeks. The data were interpreted that BMP2 produced at high levels over the entire healing time was counterproductive. (Egermann, et al. 2006 Gene Ther) In a canine defect study, high local doses were administered. After 4 weeks 800 μg/implant was found to be too high to work effectively. In a rat non-union fracture model assessed in 3 and 18 month old rats, the older rats healed more slowly than the younger rats when treated with rhBMP7, with the mechanical strength approaching that of the intact femur at 3 weeks in the young rats and not until 6 weeks in the older rats.

All of the cited references are incorporated herein by specific reference as if set forth in their entirety in this specification.

### SUMMARY OF THE INVENTION

Treatment of skeletal framework tissue, i.e. bone and cartilage tissue, is achieved in a narrow therapeutic range of HMG Co-A reductase inhibitors at the site for tissue enhancement. While any mode of administration may be used that provides the HMG Co-A reductase inhibitors for sufficient time at the site of interest, of particular interest and as preferred embodiments are the use of transdermal application and particles. By providing for a therapeutic level without using an excessive amount that must be dissipated before the therapeutic level is attained, one provides for therapeutic and economic benefits using HMG Co-A reductase inhibitors for skeletal framework enhancement.

As indicated above, bone and cartilage enhancement is achieved, using a pharmaceutical composition for topical application comprising a statin and a pharmaceutically acceptable carrier suitable for topical delivery of the statin through the skin of a subject resulting in a desired statin blood serum concentration within a short period of time.

Also, as indicated above, bone and cartilage tissue enhancement responsive to statin activity is achieved using statin containing particles in proximity to the enhancement site, where a therapeutically effective range of statin concentration is maintained at the site for a time sufficient to allow for the desired level of enhancement. Depending upon the nature of the particles, the particles may range from 100% of the statin therapeutic agent to about 10 weight % and the rate of release is controlled non-mechanically using physical and/or chemical properties. The particles are administered in accordance with a prescribed regimen adapted for the particular site and nature of the tissue enhancement activity. Rapid restoration of the tissue is achieved.

### BRIEF DESCRIPTION OF THE FIGURES

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a comparison of oral vs. dermal administration of lovastatin. Plasma lovastatin levels were measured after a single dose (a: 10 or b: 50 mg/kg). Plasma was collected at times specified after dosing. The concentration of lovastatin was estimated using the HMG-CoA reductase inhibitory assay. Values are mean±SEM (n=5).

FIG. 2 illustrates an assessment of BMD at the proximal tibiae in intact rats using Piximus bone densitometer. Measurements were obtained at the end of the five weeks. Each data point is the mean±SEM of 10 animals.

FIG. 3 illustrates the bone volume (BV/TV %) in (a) intact and (b) OVX rats treated with transdermal lovastatin (hydrophilic petrolatum) for 5 days only. Bones were removed 4 weeks after treatment ended and processed for histology. Numbers inside bar represent percentage increase compared to vehicle-treated controls. Each data point is the mean±SEM of 10 animals. p<0.05 vs. intact or OVX+vehicle.

FIG. 4 is a histomorphometric analysis of the cancellous bone of the proximal tibial metaphysis in SHAM and OVX rats after 5 day treatment with 1 mg/kg/day dermal lovastatin. Numbers inside bars represent % change from respective controls, i.e., vehicle-treated OVX rats compared to vehicle-treated SHAM rats, treated OVX rats compared to vehicle-treated OVX rats. b) Representative undecalcified sections of the proximal tibia stained with van Gieson (black and white images). Each data point is the mean±SEM of 10 animals. p<0.05 vs. sham or OVX+vehicle.

FIG. 5 illustrates histomorphometric results in SHAM and OVX rats showing structural indices of trabecular bone architecture. Numbers inside bars represent % increase compared to vehicle treated OVX rats. a) Trabecular thickness, b) trabecular number and c) trabecular separation. Each data point is the mean±SEM of 10 animals. p<0.05 vs. SHAM or OVX+vehicle.

FIG. 6 illustrates the effect of 5 day administration of dermal lovastatin on bone formation rates (BFR) in SHAM and OVX rats. Numbers inside bars represent % increase compared to vehicle treated OVX rats. Values are the mean±SEM of 10 rats.

FIG. 7 illustrates rat distal femur metaphyseal trabecular bone analysis by μCT. Representative photomicrographs showing cancellous bone in distal femoral metaphyses from 3 groups of intact rats: Vehicle treated, and lovastatin treated (transdermal) with 1 and 5 mg/kg/day for 5 days, and comparison with μCT images. Femurs were scanned using the Skyscan 1072 employing an x-ray tube voltage of 100 kV, and magnified to attain a pixel size of 10.13 μm. At this resolution the trabecular structure was accurately reconstructed. Images correspond to metaphyseal region 1-2 mm distal to the growth plate. Numbers inside bars represent % increase compared to vehicle treated rats.

FIG. 8 shows the biodistribution of lovastatin after dermal application. Comparison of hydrophilic petrolatum (HP) versus hydroalcoholic gel (HA gel). A single dose of lovastatin was administered using either formulation and AUC0-24 hr calculated using the trapezoidal rule. a) Single dermal application of lovastatin: 6.25 mg/kg. b) Lovastatin was applied dermally with a single dose of 25 mg/kg.

FIG. 9 depicts bone volume assessment of ovx rats treated five days after surgery with dermal lovastatin in hydroalcoholic gel for 5 days only with a dose scheme ranging from 0.01 to 0.5 mg/kg/day. Four weeks after the end of dosing, animals were sacrificed and bones collected for histomorphometric analysis. Numbers inside bars represent % change compared to controls. OVX decreased bone volume by 59% (compared with vehicle-treated SHAM group. Dermal treatment with lovastatin increased bone volume >40% compared to vehicle-treated OVX rats. Graph shows mean values±SEM for cancellous bone volume in proximal tibiae (n=10/group).

FIG. 10 illustrates serum osteocalcin in rats treated with dermal lovastatin for 5 days as measured twenty six days after the initial dosing. Number inside bar represents % increase compared to vehicle-treated OVX rats. Graph shows mean values±SEM (n=8/group).

FIG. 11 illustrates quantification of serum creatine protein kinase (CPK) in shamd and ovx rats treated with lovastatin in hydroalcoholic gel for 5 days. No significant changes were observed among the treated groups vs. control. Values are the mean±SEM of 10 rats.

FIG. 12 is a bar graph showing the radiographic score at 2 weeks using transdermal delivery of lovastatin as compared to higher levels administered orally using a femur fracture model.

FIG. 13 is a bar graph of the breaking force using transdermal and oral delivery of lovastatin using a femur fracture model.

FIG. 14 is a bar graph of the breaking force using lower doses of transdermal and oral delivery of lovastatin using a femur fracture model.

FIG. 15 is a bar graph of the stiffness measured 6 weeks after fracture using transdermal and oral delivery of lovastastin using a femur fracture model.

FIG. 16 is a bar graph of the lovastatin plasma concentration for transdermal and oral delivery.

FIG. 17 is a bar graph of the lovastatin plasma concentration from lovastatin nanobeads showing that the amount of lovastatin is below the limit of detection.

FIG. 18 is a bar graph of the radiographic score using nanobeads containing lovastatin at various levels of release of lovastatin.

FIG. 19 is a bar graph of the maximum strength resulting from treatment with nanobeads at various levels of release of lovastatin using a femur fracture model.

FIG. 20 is a bar graph of the work required to fracture resulting from treatment with nanobeads at various levels of release of lovastatin using a femur fracture model.

FIG. 21 is a bar graph of quantitation of cartilage growth seen in neonatal murine calvaria seen at day 14 following exposure to lovastatin. The bars are in the order from left to right of the order of treatment from top to bottom.

### DESCRIPTION OF THE EMBODIMENTS

HMG Co-A reductase inhibitors are administered, particularly in a narrow therapeutic range window, for enhancement of bone and cartilage tissue. The administration provides a biodistribution profile designed to maximize bioavailability of the HMG Co-A reductase inhibitors to the skeletal tissue while minimizing bioavailability to non-skeletal tissue. Furthermore, it is found that there is a narrow window of concentrations of therapeutic efficacy over a restricted period of time, where larger or smaller amounts administered to the host and shorter or longer periods of treatment provide for substantially diminished or no benefit to the host. In addition, by using dosages in the therapeutic window, side effects of the drug are diminished or avoided and a more economic treatment is achieved. In addition, by limiting the duration of the treatment, one avoids negative effects of the HMG Co-A reductase inhibitors occurring after prolonged treatment. Also, by controlling the duration, one further avoids side effects of the drug and economic benefits result in shorter treatment times. Therefore, the administration of the drug and the duration of the administration will be at an amount and for a time to substantially optimize the response at the site of interest, namely the site being treated to enhance the bone and/or cartilage at the site. The amount administered will vary with the mode of administration, while the time of administration will generally vary with the indication being treated and the nature of the host. Other than oral administration, primarily parenteral and inhalation, is employed to provide the HMG Co-A reductase inhibitors directly to the host system, particularly to the site of treatment, without significant uptake of the HMG Co-A reductase inhibitors by the liver.

The modes of administration may vary from any mode other than oral that provides the desired therapeutic range for a time sufficient to induce the desired degree of enhancement. While not being limited to any theoretical explanation of the observed results, it appears that the results have a Gaussian distribution, in that below the desired range, there is little tissue enhancement, while above the desired range, there is no significant increase in tissue enhancement, and, in fact, there may be less enhancement as compared to the desired range over the time of treatment. The observed results are rationalized that both osteoblasts and osteoclasts are involved in the restoration, i.e. repair and degration, of bone. Analogously, the situation with cartilage involves chondrocytes for repair and degradation. The HMG Co-A reductase inhibitors are believed to stimulate cells involved in repair, e.g. osteoblasts, while inhibiting cells involved in degradation, e.g. osteoclasts. The repair and degradation are involved in proper remodeling of the skeletal framework tissue. It is therefore believed, that the amount of the HMG Co-A reductase inhibitors and the duration of the treatment should be selected to provide for proper remodeling.

The subject method provides for substantial optimization of the usage of the HMG-CoA reductase inhibitor, resulting in substantial benefits to the host being treated. Not only does one achieve economies in using lower dosages than have heretofore been used, but repair is accelerated as compared to the higher dosages, the patient recovers more rapidly, is subject to fewer side effects of the drug, and can more rapidly assume normal activities.

In referring to tissue enhancement, the results may vary and can be most easily expressed in describing fractures. One is interested in the case of fracture of having a properly remodeled bone that is capable of withstanding weight and normal use within the shortest time. With a fracture, one can measure the degree to which the fracture has knitted together and can withstand mechanical forces, such as being weight bearing and/or responding to other mechanical stress. In addition, with X-rays one can observe the degree to which new bone formation has occurred and the shape of the site being treated. In the case of dental application, the degree to which the tooth or implant can withstand normal use can also be observed. In the case of bone fusion, one can observe the joining of the bones and the ability of the fusion to withstand stress. Other indications can be similarly analyzed Thus, while one can provide guidelines for treating various indications, the great variety of situations to which the present invention may be applied, means that there will be situations where the dosage and/or time of treatment may need to be determined empirically by observing the response to the treatment or using a model as described in the experimental section to evaluate the particular mode of treatment as compared to known modes of treatment that have provided outcomes with the indicated model.

Modes of administration are parenteral or inhalation and include injection of the drug in an appropriate form and medium, administration by a pump, transdermal administration, inhalation as available, etc. The HMG Co-A reductase inhibitors may be present in a fluid medium, solvent or non-solvent, dissolved or stably dispersed, as particles, where the particles may vary from 10 to 100% of the therapeutic agent, dispersed neat or as particles in a gel, e.g. hydrogel or temperature sensitive gel, combined with an adhesive cement, impregnated, coated or formed as a film, mesh or fiber, normally in conjunction with a carrier, particularly a polymer matrix or an inorganic matrix, particularly an osteoconductive inorganic matrix, e.g. apatite, or the like.

### General Considerations for Administration of HMG-CoA Reductase Inhibitor

The mode of administration should provide a therapeutic amount of the HMG Co-A reductase inhibitor for sufficient time to provide the desired enhancement of the skeletal framework tissue, particularly remodeling of the structure being treated. As a rough equivalency, treatment levels are in the ratio of 1:4:200 for mouse, rat and human. The amount of the HMG Co-A reductase inhibitors is the bioavailable amount, as drug that is not available to the site of interest, e.g. sequestered by an organ or subject to rapid degradation, will not provide the desired effect. Dosage levels will generally be in the range of about 0.01 to 10, more usually 0.025 to 5 and preferably 0.05 to 2.5 mg/kg/day, where the amount may be modified to some degree when treating a human host. Generally, the amount of HMG Co-A reductase inhibitor delivered to the rat host will be in the range of about 0.1 to 5, usually 0.1 to 2 mg/kg/day, with modifications as appropriate in accordance with the particular mode of treatment and the indication. For a human, the range will be about 5 to 250 μg/day. Desirably during the course of treatment, the blood concentration of the HMG Co-A reductase inhibitor should be in the range of about 0.5 to 5, more usually 1 to 5 ng/ml. The treatment duration for humans will generally be greater than 1 day, usually greater than 2 days, more usually greater than about 5 days, desirably up to and including 10 days and not more than about 65 days, usually not more than about 25 days, and more usually not more than about 15 days, generally not more than 10 days. Treatment is terminated when further treatment results in no tissue enhancement or deleterious effects, such as side effects of the drug and diminished positive or negative osteogenic response to the drug.

Until there has been substantial use of the subject methodology, monitoring of the patient will be valuable to ascertain the optimum dosage and optimum duration. Once experience has been obtained with a specific formulation and particularly with a specific indication that experience may then be used in future therapies.

In a specific situation, depending on the form of treatment, one can determine the efficacy as to dosage and duration by using a rat model as described in the Experimental section. In light of the manifold forms in which the HMG Co-A reductase inhibitors can be provided, the media employed and the manner of administration, there can be situations where one would wish to use the animal model to verify the efficacy of a particular mode of treatment.

Various HMG-CoA reductase inhibitors may be used and as new HMG-CoA reductase inhibitors or their analogs are developed they are also included. Statins known today are described in S. E. Harris, et al. (1995) Mol Cell Differ 3, 137; G. Mundy, et al. Science (1999) 286, 1946; and U.S. Pat. Nos. 6,022,887; 6,080,779 and 6,376,476, whose disclosure of statins is specifically incorporated herein by reference. Illustrative statins include lovastatin, pravastatin, velostatin, simvastatin, fluvastatin, cerivastatin, mevastatin, dalvastatin, fluindostatin, rosuvastatin and atorvastatin. Also included are prodrugs of these statins, their pharmaceutically acceptable salts, e.g. calcium, etc. The preparation of these compounds is well known as set forth in numerous U.S. Pat. Nos. 3,983,149; 4,231,938; 4,346,227; 4,448,784; 4,450,171; 4,681,893; 4,739,073; and 5,177,080. Since these compounds are also generally commercially available, they can be purchased as required.

The subject therapeutic regimens allow for treatment of a mammalian species host (e.g. human) which suffers from a skeletal framework disorder requiring administration of a HMG Co-A reductase inhibitor. Generally, the patient is a human predisposed to, or suffering from a skeletal (bone or cartilage) disorder such as Achondroplasia, Acquired Hyperostosis Syndrome, Acrocephalosyndactylia, Arthritis, Arthritis, Juvenile Rheumatoid, Arthritis, Rheumatoid, Arthrogryposis, Arthropathy, Neurogenic Bone Diseases, Cartilage Diseases, Cleidocranial Dysplasia, Clubfoot, Compartment Syndromes, Craniofacial Dysostosis, Craniosynostoses, Dwarfism, Ellis-Van Creveld Syndrome, Enchondromatosis, Exostoses, Fibrous Dysplasia of Bone, Fibrous Dysplasia, Polyostotic, Flatfoot, Foot Deformities, Freiberg's Disease, Funnel Chest, Goldenhar Syndrome, Hallux Valgus, Hip Dislocation, stress fractures, Congenital Hyperostosis, Intervertebral Disk Displacement, Joint Diseases, Kabuki Make-Up Syndrome, Klippel-Feil Syndrome, Langer-Giedion Syndrome, Legg-Perthes Disease, Lordosis, Mandibulofacial Dysostosis, Melorheostosis, Musculoskeletal Abnormalities, Myositis Ossificans, Osteitis Deformans, Osteoarthritis, Osteochondritis, Osteogenesis Imperfecta, Osteomyelitis, Osteonecrosis—Osteopetrosis Osteoporosis—Poland Syndrome, Rheumatic Diseases, Russell Silver Syndrome, Scheuermann's Disease, Scoliosis, Sever's Disease/Calceneal Apophysitis, Spinal Diseases, Spinal Osteophytosis, Spinal Stenosis, Spondylitis, Ankylosing, Spondylolisthesis, Sprengel's Deformity, Tennis Elbow, Thanatophoric Dysplasia, bone deficit conditions, compromised skeletal healing, non-union fractures, closed or simple fractures, open or compound fractures, dental deficit conditions, dental implant fixation, orthopedic fixation, spinal fusion, cartilage deficit conditions.

### Transdermal Application

In a preferred mode for providing the desired treatment as to concentration and duration, where one can achieve long term release while maintaining a relatively constant dosage to the site of interest, topical application can be employed. As indicated above, particles can be used in the topical applications described below, as well as dispersed HMG-CoA reductase inhibitor. The amount of HMG-CoA reductase inhibitor administered will generally be from about 0.05 to 20 mg/kg/day, more generally 0.05 to 10 mg/kg/day, usually from about 0.1 to 10 mg/kg/day, preferably in the range of about 0.1 to 2.5 mg/kg/day. This intends that this amount will be bioavailable to the site of interest, where greater amounts may be required where the application is distal to the site of interest or applied over a large surface.

As used herein, the phrase “topical application” describes application onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed hereinbelow, the compositions of the present invention may be formulated into any form typically employed for topical application.

Hence, the pharmaceutical compositions of the present invention can be, for example, in a form of a cream, an ointment, a paste, a gel, a lotion, milk, a suspension, an aerosol, a spray, foam, a shampoo, a hair conditioner, a serum, a swab, a pledget, a pad, a patch and a soap. Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight. Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like. Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information. Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information. Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration. Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment. Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present invention may, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

The pharmaceutical composition of the present invention will be formulated to provide the indicated therapeutic level of HMG Co-A reductase inhibitor as indicated above. The amount of HMG Co-A reductase inhibitor may vary widely depending upon the specific formulation, the site as which the formulation is applied as compared to the site of interest requiring treatment, the area to which the formulation is applied, and the like. For the most part, the amount of the pharmaceutical composition ranges between about 0.1 mg and about 10 mg/cm² of the biological surface per day.

When provided as a cream or ointment, the pharmaceutical composition of the present invention typically includes HMG Co-A reductase inhibitor and a hydrophilic petrolatum, aqueous alkanolic gel or a pluronic lecithin organogel (PLO).

An aqueous alkanolic gel with a carbomer-based formulation can contain, for example, 60% ethanol, <40% ddH₂0, 1% Carbomer polymer of either 940 or 980, 0.5% cholesterol, 0.1% BHA, 3% TTA and HMG Co-A reductase inhibitor. Such a gel can be manufactured by slowly (drop wise) adding (while stirring) H₂0 (1 ml) to a Carbomer 940/H₂0/triethanolamine mixture and slowly (drop wise) mixing in enough ethanol to make 10 ml of product. The pH of the final mixture should be >4.5. The final product is aliquoted and sealed and protected from light.

For pluronic gels selected components are combined and delivered in a topical vehicle, preferably pluronic lecithin organogel (PLO). Methods of topical application are as cream, gel, ointment, spray or patch, especially by iontophoresis delivering the components through an iontophoretic patch.

A preferred composition includes a HMG Co-A reductase inhibitor such as lovastatin and a topical gel preparation. The selected HMG Co-A reductase inhibitor is incorporated into pluronic lecithin organogel (PLO) to facilitate transdermal administration.

These components are mixed in a controlled environment. Precautionary measures should protect pharmaceutical workers from active ingredients that may become airborne or topically absorbable. In the United States, OSHA complaint safety procedures should be followed.

The composition can include a pharmaceutically acceptable liquid carrier which includes a biphasic complex of lecithin and organogel, for molecular egression across the epidermis to the superficial and deep dermis where vascular structures reside.

PLO is a phospholipid liposomal micro emulsion used for transdermal drug administration. PLO has two phases:

(i) An oil Phase: the oil phase is lecithin/isopropyl palmitate solution. Lecithin rearranges the horny layer of the skin. Isopropyl palmitate is a solvent and penetration enhancer. Sorbic acid is a preservative.

(ii) A water Phase: the water phase is a pluronic gel. Pluronic f127 NF is a commercial surfactant. Potassium sorbate NF is a preservative. Purified water is a solvent. The active agents are incorporated into the PLO gel and a stable emulsion is formed through sheer force. The concentration of the active agents in the formulation may be adjusted as to obtain the optimal therapeutic response.

A composition of the active agents and carrier is prepared according to the following procedure. First, HMG Co-A reductase inhibitor is solubilized; it is then combined with the lecithin/isopropyl palmitate solution and mixed well. Pluronic F127 is then added as a 20% gel in small increments to a final desired volume. The composition is then mixed at high speed in an electric mortar and pestle to form a smooth creamy gel.

Once prepared, the topical HMG Co-A reductase inhibitor formulation of the present invention can be administered topically either by the patient or by a heath care provider. When the dosage form is a topical cream-gel suspension or topical patch methodology, it may contain preservatives, stabilizers, emulsifiers or suspending agents, wetting agents, salts for osmotic pressure or buffers, as required. When the dosage form is as a pressurized spray or aerosol, the solution is contained in a pressurized container with a liquid propellant such as dichlorodifluororo methane or chlorotrifluoro ethylene. If administered from a pump container, the solution will include a buffer salt solution with preservatives, stabilizers, emulsifiers or suspending agents, wetting agents, and salts for osmotic pressure or buffers, as required.

When the composition is administered in the form of topical gel-cream, spray, or topical iontophoresis gel patch, the time of repeat application will vary from every six to twelve hours for the gel-cream and spray to several days for the topical iontophoresis gel-patch delivery methods. Occlusion with a barrier ointment or physical barrier such as hypoallergenic membrane may also be practiced after topical application of the gel-cream or spray to increase efficacy and penetration of the pharmaceutical.

When provided as a patch or any other transdermal delivery device, the pharmaceutical composition of the present invention includes a HMG Co-A reductase inhibitor, such as lovastatin. A preferred patch formulation would be a single-layer drug-in-adhesive system where the HMG Co-A reductase inhibitor in directly included within the skin-contacting adhesive. Preferred concentration ranges would be such that the patch delivers sufficient HMG Co-A reductase inhibitor for an effective concentration at the site of interest. Subject to the previously indicated caveats, this will generally fall between 0.01-1 mg/kg per day.

When provided as an aerosol or other transmucosal delivery device, the pharmaceutical composition of the present invention typically includes a HMG Co-A reductase inhibitor such as lovastastin. Preferred aersol or other transmucosal delivery device would include technologies such as Metered Dose Inhalers (MDI) such as asthma inhalers which mediate the airways but not deep into the lungs, Nebulisers which would permit a fine liquid spray, dry Powder Inhalers (DPI) or liquid Micro Droplet Inhalers. Alternative dosage forms for transmucosal or buccal delivery would include delivery systems such as mouthwashes, erodible/chewable buccal tablets, and chewing gums Bioadhesive buecut films/patches and tablets fabricated using various geometries either as a single-layer device, from which drug can be released multidirectionally or a device that has a impermeable backing layer on top of the drug-loaded bioadhesive layer where drug loss into oral cavity can be greatly decreased. Another device configuration can include a unidirectional release mechanism thus minimizing drug loss and enhancing drug penetration through the buccal mucosa.

Since HMG Co-A reductase inhibitors lower production of cholesterol which is a major component of cells including dermal and mucosal cells, topical administration of a HMG Co-A reductase inhibitor can lead to cholesterol depletion in such cells which could lead to reduced permeability of HMG Co-A reductase inhibitor. Thus, in order to increase the penetration of HMG Co-A reductase inhibitor through the biological surface, the pharmaceutical composition of the present invention preferably further includes cholesterol at a concentration of 0.1-1% by weight.

The pharmaceutical composition of the present invention can also include a penetration enhancer such as simple alkyl esters, phosopholipids, terpenes, supersaturated solutions, ultrasound, organic solvents, fatty acids and alcohols, detergents and surfactants, D-limonene, β-cyclodextrin, DMSO, polysorbates, bile acids, N-methyl pyrrolidine, polyglycosylated glycerides, 1-dodecylazacycloheptan-2-one (Azone®), cyclopentadecalactone (CPE-215®), alkyl-2-(N,N-disubstituted amino)-alkanoate ester (NexAct®), 2-(n-nonyl)-1,3-dioxolane (SEPA®), Carbomer polymers, pluronic gels, lecithin, tri-block copolymers such as Pluronic 127 as well as stabilizers or neutralizers such as, BHA, benzoic acid, sodium hydroxide, potassium hydroxide triethanol Amine triethyl amine, other diluents in alkaline form, such as water, ethanol, and the like.

The present invention further encompasses processes for the preparation of the pharmaceutical compositions described above. These processes generally comprise admixing the active ingredients described hereinabove and the pharmaceutically acceptable carrier. In cases where other agents or active agents, as is detailed hereinabove, are present in the compositions, the process includes admixing these agents together with the active ingredients and the carrier. A variety of exemplary formulation techniques that are usable in the process of the present invention is described, for example, in Harry's Cosmeticology, Seventh Edition, Edited by J B Wilkinson and R J Moore, Longmann Scientific & Technical, 1982, Chapter 13 “The Manufacture of Cosmetics” pages 757-799 as well as in Pharmaceutical development and clinical effectiveness of a novel gel technology for transdermal drug delivery Alberti, I. et al Expert Opinions in Drug Delivery 2: 935-50, 2005, Mucosal drug delivery: membranes, methodologies, and applications, Song, Y et al Critical Reviews Therapeutic Drug Carrier Systems 21: 195-256, 2004 and Drug delivery systems: past, present, and future Mainardes, R. M. et al. Current Drug Targets 5: 449-55, 2004.

### Particle Administration

One form of HMG Co-A reductase inhibitors of particular interest is in the form of small particles, particularly micro- or nanoparticles. The compositions comprise particles that as a result of the low solubility of statins in aqueous media dissolve over time or slow release particles, nano or micro, comprising at least one HMG-CoA reductase inhibitor. The particles can be formed in any convenient manner to provide for homogeneous, substantially homogeneous or heterogeneous size distribution. For the most part, the particles are administered to the site of interest in an appropriate vehicle and maintained at the site of interest for sufficient time to provide tissue enhancement.

Generally, the particles will release the HMG-CoA reductase inhibitor at a rate in the range of about 0.5 to 2.5, more usually in the range of about 1 to 2, μg/day. By site of interest is intended the site where there is to be enhancement of bone and/or cartilage tissue, generally being within about 5 cm of the site, so as to release the HMG-CoA reductase inhibitor directly in association with the tissue being treated. However, there can be instances where the particles will be administered at a different site and the effect will rely on the release of the HMG-CoA reductase inhibitor from the particles where the released HMG-CoA reductase inhibitor is transported to the site of interest.

The particles provide for a continuing therapeutic amount of the HMG-CoA reductase inhibitor over the prescribed treatment period. The particles administered provide for a relatively uniform release of the HMG-CoA reductase inhibitor over a predetermined period of time. By appropriate selection of particle composition and amount of particles administered, the period of time at which the site of interest is exposed to the drug at a therapeutic level provides for controlled tissue enhancement.

The particles are prepared to allow for the slow release of the HMG-CoA reductase inhibitor at a predetermined rate, so that over the period of treatment, the level of HMG-CoA reductase inhibitor at the site is sufficient to provide cell activation and tissue enhancement. The particles may vary from substantially homogeneous HMG-CoA reductase inhibitor, as pure drug particles, varying from completely crystalline to completely amorphous and/or vitrified, to particles with the HMG-CoA reductase inhibitor as small particles interspersed in a carrier, a single core, HMG-CoA reductase inhibitor molecules dispersed in a carrier, such as a hydrogel, which may include a rate controlling surface membrane.

The release of the HMG-CoA reductase inhibitor from the particles is controlled by non-mechanical means, namely physical and/or chemical phenomena. These phenomena include osmosis, dissolution, hydrolysis, degradation, salvation, erosion, etc. where the HMG-CoA reductase inhibitor is slowly released into the environment of the site of interest. Normally, there is a curve where initially the amount of HMG-CoA reductase inhibitor released increases to a maximum, followed by a low diminution of the amount of HMG-CoA reductase inhibitor released per unit time interval, and then frequently there is a breakdown of the particle where the remaining HMG-CoA reductase inhibitor is released over a short period of time. The average release rate will usually be between about 0.5 to 20%, more usually between about 5 to 20% to breakdown of the particles, based on a 24 h time period. Desirably, the residue at breakdown will be less that 20% of the original amount of HMG-CoA reductase inhibitor, preferably less than about 15%.

Depending upon the nature of the particles and the manner of their formation, one may have a substantially homogeneous sized composition of particles or a heterogeneous sized composition of particles, where the different sized particles will have different release profiles over time to provide the desired range of HMG-CoA reductase inhibitor concentration over the therapeutic time interval. The size dispersion may have two or more groups of sized particles, where each group will have at least about 75 weight % of particles of a size within 50% of the median size. Alternatively, one may have a relatively uniform narrow range or broad range of particle sizes.

The particles are biocompatible and conveniently bioresorbable, where particles comprising a carrier will normally be biodegradable. The particles will usually leave no residue and will result in minimal inflammation, if any, at the site being treated. At least 60 weight %, more usually at least about 70 weight % of the particles will be in the size range of about 0.001 to 100 μm, and generally at least about 60 weight %, more usually at least about 75 weight % will be within about 35%, preferably within about 20% of the median size particle for a homogeneous sized composition. (In referring to size one is considering the mean diameter.) Where the solid drug is milled or ground, one will usually have a heterogeneous mixture of particles where more than 50 weight %, more usually more than 60 weight %, will be within 50% of the median size of the particles. If desired, the particles may be sized using screens or other method for providing particles in a particular range, where only particles in the particular range are used, or combinations of particles of the different ranges may be used. For a heterogeneous composition, there may be 1, 2 or 3 different groups having narrow size ranges, where the median size of any one group will usually be not more than about 100 times the next smaller median size, more usually not more than about 50 times the next smaller median size. The weight ratio of the groups will depend upon the release profile, where the smaller particles will generally release more of the HMG-CoA reductase inhibitor in the early period, while the larger particles will release the HMG-CoA reductase inhibitor later than the smaller particles.

One may use nanoparticles or microparticles, which will normally involve a carrier, where these groups of particles will fall into different size ranges. The nanoparticles will generally be in the range of about 1 to 50, more usually 5 to 25 nm, with the distribution as indicated above. The microparticles will generally be in the range of about 1 to 200 μm, more usually in the range of about 5 to 100 μm, with the distribution as indicated above. Only a few large particles can unduly distort the weight/size distribution. It should be understood that in the event of a few outliers the numbers given may be somewhat off and such outliers should not be considered in the distribution, as they generally will not exceed 10 weight % of the composition and will be at least about 1.5 times greater than the largest particle coming within the distribution.

The particle composition will be chosen to provide a continuous level of HMG-CoA reductase inhibitor at the site of interest, based on the area of the site to be treated, of about 10⁻⁵-10⁻³ mg/mm²-day. More than one injection may be involved, so that the particle composition provides for the predetermined duration. The total number of days has been indicated previously. Where successive injections are employed, there may be periods of overlap, where the total amount of HMG-CoA reductase inhibitor being released for a short period, generally less than about 12 hours, more usually less than about 6 hours, is in excess of the amount indicated above. In order to achieve extended lengths of time while maintaining a therapeutic level, one or more administrations of the particles may be required, usually not more than daily and preferably not more than at intervals of about 3 days, more usually not more than at intervals of about 7 days, desirably at intervals not more than about 10 days, and may be single doses at intervals of 30 or more days.

The HMG-CoA reductase inhibitor can be prepared neat as a vitreous or crystalline particle. The particles can be either micro or nano as the sizes have been described above, and may be amorphous or crystalline, where the crystallinity can vary from about 0 to 100%. For slower release, the at least substantially crystalline particles will be used, where for more rapid release more of the amorphous drug will be present. One may also use powders where the pure drug is milled or ground to a predetermined size distribution. Various mechanical methods may be employed to provide the desired powder size distribution. Generally, large clumps are avoided, so that a relatively narrow size distribution is obtained, conveniently falling within the size range of the nano- or microparticles, but may also include fines that may fall outside those ranges. The fines will generally be less than about 20, usually less than about 10 weight % of the composition.

A wide range of particle compositions may be employed depending upon the nature of the site to be treated, the desired release profile, the amount of HMG-CoA reductase inhibitor required for the treatment, the time interval for providing the therapeutic level of HMG-CoA reductase inhibitor and the permitted volume of the particles at the site of interest.

One or more compositions may be used in the particle matrix, where one composition may be dispersed in the other, form a partial or complete coating of the other composition, or the like and the HMG-CoA reductase inhibitor may be an internal particle, e.g. core, or dispersed in one or more of the compositions to provide the desired slow release profile. The polymers that find use include both addition polymers and condensation polymers. The polymeric compositions that find use are biocompatible polymers that are normally resorbable, particularly biodegradable, which biodegradable polymers include: polymers of water soluble hydroxylaliphatic acids, particularly α-hydroxyaliphatic acids, oxiranes, vinyl compounds, urea derivatives, saccharides, orthoesters, anhydrides, hydrogels, etc. Compositions that may find use include polylactic acid (PLA) either a pure optical isomer or mixture of isomers, polyglycolic acid (PGA), copolymers of lactic acid and its optically active forms and glycolic acid (PGLA), copolymers of lactic acid and caprolactone, copolymers of glycolic acid and caprolactone, terpolymers of lactic acid, glycolic acid and caprolactone, polycaprolactone; polyhydroxybutyrate-polyhydroxyvalerate copolymer; poly(lactide-co-caprolactone); polyesteramides; polyorthoesters; poly ω-hydroxybutyric acid; and polyanhydrides, block copolymers of the preceding with poly(ethylene glycol), or block copolymers of any combination of the preceding polymers.

Polymers which are generally biocompatible but not biodegradable include polymers such as: polydienes such as polybutadiene; polyalkenes such as polyethylene or polypropylene; polymethacrylics such as polymethyl methacrylate or polyhydroxyethyl methacrylate; polyvinyl ethers; polyvinyl alcohols; polyvinyl chlorides; polyvinyl esters such as polyvinyl acetate; polystyrene; polycarbonates; poly esters; cellulose ethers such as methyl cellulose, hydroxyethyl cellulose or hydroxypropyl methyl cellulose; cellulose esters such as cellulose acetate or cellulose acetate butyrate; polysaccharides; and starches, alkyl cyanoacrylates, polyurethanes.

Crosslinked biocompatible but not biodegradable polymers include hydrogels prepared from polyvinyl acetate (PVA), polyvinyl pyrrolidone, polyvinyl alcohol (xl-PValc), polyalkyleneoxides, particularly polyethylene oxide (PEG), etc., where the polymers may be cross-linked, modified with various groups, such as aliphatic acids of from 2 to 18 carbon atoms, alkyleneoxy groups of from 2 to 3 carbon atoms, and the like. The polymers may be homopolymers, co-polymers, block or random, may include dendrimers, etc.

Of particular interest are the polymers and copolymers of α-hydroxyaliphatic carboxylic acids of from 2-3 carbon atoms. Lactide/glycolide polymers for drug-delivery formulations are typically made by melt polymerization through the ring opening of lactide and glycolide monomers. Some polymers are available with or without carboxylic acid end groups. When the end group of the poly(lactide-co-glycolide), poly(lactide), or poly(glycolide) is not a carboxylic acid, for example, an ester, then the resultant polymer is referred to herein as blocked or capped. The unblocked polymer, conversely, has a terminal carboxylic group. The biodegradable polymers herein can be blocked or unblocked. In a further aspect, linear lactide/glycolide polymers are used; however star polymers can be used as well. Low or medium molecular weight polymers are used for drug-delivery where resorption time of the polymer and not material strength is important. The lactide portion of the polymer has an asymmetric carbon. Commercially racemic DL-, L-, and D-polymers are available. The L-polymers are more crystalline and resorb slower than DL-polymers. In addition to copolymers comprising glycolide and DL-lactide or L-lactide, copolymers of L-lactide and DL-lactide are available. Additionally, homopolymers of lactide or glycolide are available.

In the case when the biodegradable polymer is, poly(lactide), poly(glycolide), or poly(lactide-co-glycolide), in the latter case the amount of lactide and glycolide in the polymer can vary. In a further aspect, the biodegradable polymer contains 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In a further aspect, the biodegradable polymer can be poly(lactide), 95:5 poly(lactide-co-glycolide) 85:15 poly(lactide-co-glycolide), 75:25 poly(lactide-co-glycolide), 65:35 poly(lactide-co-glycolide), or 50:50 poly(lactide-co-glycolide) where the ratios are mole ratios.

Polymers that are useful for the present invention are those having an intrinsic viscosity of from 0.15 to 2.0, 0.15 to 1.5 dL/g, 0.25 to 1.5 dL/g, 0.25 to 1.0 dL/g, 0.25 to 0.8 dL/g, 0.25 to 0.6 dL/g, or 0.25 to 0.4 dL/g as measured in chloroform at a concentration of 0.5 g/dL at 30° C. In a further aspect, when the biodegradable polymer is poly(lactide-co-glycolide), poly(lactide), or poly(glycolide), the polymer has an intrinsic viscosity of from 0.15 to 2.0, 0.15 to 1.5 dL/g, 0.25 to 1.5 dL/g, 0.25 to 1.0 dL/g, 0.25 to 0.8 dL/g, 0.25 to 0.6 dL/g, or 0.25 to 0.4 dL/g as measured in chloroform at a concentration of 0.5 g/dL at 30° C.

Other forms of particles may be used, such as a core coated with a mixture of the HMG-CoA reductase inhibitor and an adhesive or other polymeric matrix. For example, an inorganic core may be used, such as a calcium phosphate, e.g. tricalcium phosphate, or other osteoconductive or osteoinductive material, or an organic core, such as collagen or other protein, organic polymer, etc., in the form of fibers, mesh, etc.

Among gels, of particular interest are thermoreversible gels that at a lower temperature are readily flowable and injectable, while at an elevated temperature become more rigid. This can be achieved, for example with the dispersion of the HMG-CoA reductase in mucoadhesive compositions, such as Noveon, particularly combined with a thermosensitive material, such as Pluronic F-127. Exemplary compositions are described in Tirnaksiz and Robinson, Pharmazie 2005, 60(7):518-23. (This reference is specifically incorporated by reference in its entirety.)

Where the HMG-CoA reductase inhibitor is mixed with a matrix, the amount of HMG-CoA reductase inhibitor will usually not exceed 95 weight %, frequently not exceed 60%, more usually not exceed 50 weight %, and will usually be not less than about 10 weight %, more usually not less than about 20 weight %. (The particles may have other components, so that the weight percents are based on just the two components, the HMG-CoA reductase inhibitor(s) and the matrix.) Where more than one polymer is used, each polymer will be present in at least 1 weight % of the particle, more usually at least about 5 weight % of the particle. Of course, polymer coatings that may be applied for numerous different reasons may be less than 1%, where the polymer coating serves to enhance the mechanical integrity of the particles, reduce abrasion, reduce deliquescence or efflorescence, ease of handling and flowing, control the rate at which the drug is released from the particle, etc.

The weight ratio of HMG-CoA reductase inhibitor to polymer will be in the range of about 0.1-20:1, more usually in the range of about 0.25-1.5:1, being consistent with the percentages indicated above.

The number of particle compositions and methods of preparation of particles are legion. Illustrative patents and patent applications include U.S. Pat. Nos. 4,687,660; 5,128,798; 5,427,798; and 6,510,430 and U.S. application nos. 2005/0165203; 0208134; 0255165; 0287114; 0287196; and 2006/0057222, and references cited therein. Textbooks that describe the considerations in selecting the compositions and preparing the particles include: Organic Chemistry of Drug Design and Drug Action, Richard B. Silverman, 1992; Drug Delivery: Engineering Principles for Drug Therapy, W. Mark Salzman, 2001 and Pharmacokinetics and Metabolism in Drug Design (Methods and Principles in Medicinal Chemistry) Dennis A. Smith, et al., 2001. For the most part, the HMG-CoA reductase inhibitor and polymer matrix will be mixed together, usually in the presence of a solvent. Dropwise addition of the HMG-CoA reductase inhibitor to the matrix material may be used. After removing the solvent, the particles may be washed and sized. Other additives that may be used in the preparation of the particles include detergents, particular polymeric detergents, such as poly(vinyl alcohol)-partially hydrolyzed, e.g. 4-90 mol percent.

The particles can be used as a flowable mixture in a low viscosity medium, may be sintered or agglomerated to be formed into a porous mass or form, which may be further formed depending upon the site at which the particles are to be applied, may be introduced into bone cement materials, or the like. The particles can be joined to form the porous mass or form in a variety of ways. Partial solvents or softening agents may be used that soften the particle matrix, resulting in the particles becoming joined. Conveniently, the particles may be packed in a vessel or container providing a desired form or provide a form that can be further modified and the partial solvent passed through the packing to soften the surfaces of the particles. The particles are then repeatedly washed with a non-solvent in which the partial solvent is soluble to remove the partial solvent and recreate the solid surface of the particles. Alternatively, the particles may be sintered at a mild temperature, generally under 60° C. whereby the surface is softened and the particles become joined.

The particles may be formed into the porous mass by themselves or in conjunction with other materials, that are conveniently of the size range indicated for the HMG-CoA reductase inhibitor particles and have the appropriate properties for forming the porous mass, e.g. having a composition or polymeric matrix the same as or responding in the same way to the treatment as the particles containing the HMG-CoA reductase inhibitor. These other particles may include osteoinductive and/or osteoconductive materials, such as the calcium phosphates, hydroxyapatites, or other desirable additives. Sintering conditions will depend to a substantial degree on the desired degree of porosity, the material(s) used for making the particles, the effect of sintering on the release of the HMG-CoA reductase inhibitor, and the like.

Where the particles are present in a matrix or form that provides structure, the particles may be mechanically anchored in position. Conveniently a bone or tendon anchor may be used that holds the particles in close juxtaposition to the site being treated.

Formed structures may be used where the HMG-CoA reductase inhibitor is present in particles, molecularly dispersed, or provided in a structure, where the structure is impregnated, the HMG-CoA reductase inhibitor is imbedded in the structural material or coated onto the structural material. These structures may be formed to fit into the site of interest for treatment. The structures allow for release of the HMG-CoA reductase inhibitor at the desired rate by the manner in which the HMG-CoA reductase inhibitor is involved with the structure or coatings or other means can be used to control the rate of release of the HMG-CoA reductase inhibitor.

Other active components may be included in the particles or in the medium in which the particles are dispersed. Of interest are those agents that promote tissue growth or infiltration, such as growth factors. Exemplary growth factors for this purpose include epidermal growth factor (EGF), fibroblast growth factor (FGF), platelet-derived growth factor (PDGF), transforming growth factors (TGFs), parathyroid hormone (PTH), leukemia inhibitory factor (LIF), insulin-like growth factors (IGFs) and the like. Agents that promote bone growth, such as bone morphogenetic proteins (U.S. Pat. No. 4,761,471; PCT Publication WO 90/11366), osteogenin (Sampath et al. Proc. Natl. Acad. Sci. USA (1987) 84:7109-13) and NaF (Tencer et al. J. Biomed. Mat. Res. (1989) 23:571-89) are also contemplated. However, for the most part these compounds will not be included in the particles, as the proteins create difficulties in formulation and control of their release.

Other active components that may be included are those that are osteoconductive and osteoinductive, such as alloplasts, demineralized bone, hydroxyapatite, calcium phosphate, ceramics, tricalciumphosphate, collagens, proteoglycans, chitosans, etc., as well as autografts and allografts. These compositions may serve as scaffolds in the modeling of the tissue. To the extent these are used, they will be used as auxiliary agents to the primary treatment. These auxiliary agents may be administered separately from the subject particles or together admixed with the subject particles.

Methods of administration of the particles include injection, surgical placement, where the surgical implacement may be a preformed disc or shaped material, injection of a congealing system that may undergo transformation from an injectable liquid to a semisolid or solid structure by changes in temperature, pH, ionic strength, osmotic loss of water or solvent. etc. The amounts that are used of these auxiliary materials may be conventional or reduced by half or more in light of the activity of the subject particles.

In addition, in conjunction with the particles, glues may be used that maintain the particles at the site of administration. In some instances, the composition of the particle matrix may serve to bind the particles to the site, so that additional adhesive materials will not be necessary. Depending upon the nature of the site, such as a fracture, introduction of a prothesis, tooth cavity, etc., biological adhesives may serve as useful adjuncts. Bioadhesives include Bioglue, cyanoacrylates, fibrin, transglutaminase, collagen, hyaluronic acid, fibrin, etc. The amounts of the bioadhesives will depend on the particular site of interest and be used in conventional manners, generally in the ranges indicated above for the polymers. The bioadhesives may be used as the polymeric matrix or in combination with the polymeric matrices indicated above.

Ancillary materials that may be included in the medium and/or the particles include antioxidants, antibiotics, anti-inflammatories, immunosuppressors, preservative, pain medication, other therapeutics, and excipient agents.

Generally, the particles will be dispersed in a flowable medium, dispersion, slurry, etc., where the viscosity of the particle-containing medium allows for its application to the site of interest by a convenient means. For a liquid medium, saline, phosphate buffered saline, glycols, polyalkyleneoxy compounds, combinations thereof or other pharmaceutically acceptable carrier may be employed that does not cause deterioration of the particles. Desirably, the particles should have less than about 1 weight % solubility in the medium, more desirably less than about 0.5 weight %. In other situations, a thixotropic gel, dispersion, paste, chitosans, collgen gels, proteoglycans, fibrin and fibrin clots, may be employed. Thickening agents include cellulosic polymers and their derivatives such as methylcellulose, xanthan gums and their derivaties, polyacrylamides, alginate, collagens, cyanoacrylates, hyaluronic acid, mucin and other polypeptide biopolymers, chondroitin sulfate, glucosamines, pluronic polymers, keratin sulfate, dermatan sulfate, etc.

For injection of the particles, the injection volume will usually be in the range of 20 to 2000 μt, more usually in the range of about 100 to 1000 μl. The concentration of particles will generally be in the range of about 0.01 to 50 mg/ml, more usually in the range of about 0.1 to 25 mg/ml. For placement of a structured form, the form will be associated with the site of interest, being shaped appropriately for the site as in known in the field.

Various modes of administration of the particles may be used depending upon the site of interest, whether the skin is breached so the site is directly available, the nature of the treatment, etc. Where the skin is intact covering the site of interest, usually the composition will be administered by injection, using a needle of sufficient size to allow for ready passage of the particles. Where the site is available, the subject particle compositions may be directly applied to the site using syringes, surgical implantation, applied as dry particles, pumps, aerosol injection, topical application, etc.

The following examples are offered by way of illustration and not by way of limitation.

### Materials and Methods

### Transdermal

### Transdermal Study 1

Chemicals

Lovastatin was obtained from Stason Pharmaceuticals Incorporated (Irvine, Calif.). HMG-CoA, triethanolamine (TEA), demeclocycline, dimethyl sulfoxide (DMSO) and calcein were purchased from Sigma-Aldrich, (St Louis, Mo.). Glutaryl-3-[14C] HMG-CoA was purchased from Amersham Biosciences, (Piscataway, N.J.), NADPH and Dithiothreitol (DTT) from Calbiochem, (San Diego, Calif.). Methylcelullose was obtained from ICN, (Aurora, Ohio); hydrophilic petrolatum from Ambix Laboratories, (East Rutherford, N.J.); Carbomer 940 from Noveon, Inc., (Cleveland, Ohio); Cholesterol NF and butylated hydroxyanisole NF (BHA) from PCCA (Houston, Tex.). AG1-X8 resin and Poly Prep columns were obtained from Bio-Rad Laboratories (Hercules, Calif.), ketamine from Fort Dodge Animal Health, Wyeth (Madison, N.J.) Domitor and Antisedan from Pfizer (New York, N.Y.); Osteocalcin kit from Biomedical Technologies Inc. (Stoughton, Mass.)

Measurement of HMG-Co-A Reductase Activity

Plasma concentrations of lovastatin equivalents after a single dose were measured at several time points using a modification of the well-described HMG-CoA reductase inhibition assay[Germershausen J I, Hunt V M, Bostedor R G, Bailey P J, Karkas J D, Alberts A W (1989) Tissue selectivity of the cholesterol-lowering agents lovastatin, simvastatin and pravastatin in rats in vivo. Biochem Biophys Res Commun 158: 667-675.]. The soluble rat liver HMG-CoA reductase used in this assay was prepared from rat liver microsomes [Heller R A, Gould R G (1973) Solubilization and practical purification of hepatic 3-hydroxy-3-methylglutaryl coenzyme a reductase. Biochem Biophys Res Commun: 50: 859-865.]. Plasma was withdrawn from the rats after a single dose of lovastatin administered orally or dermally at 1, 3, 6 and 24 hours. The concentration of the drug was determined by comparing the amount of inhibitory activity in the plasma of treated rats to a standard curve generated by adding the active open ring form of lovastatin to normal rat plasma. This is a standard method of studying the pharmacokinetics/pharmacodynamics of lovastatin because this drug reportedly has several active metabolites [14-16]. The area under the plasma concentration-time curve (AUC0-24 hr) of lovastatin equivalence was calculated using the trapezoidal rule for both oral and dermal application of lovastatin. For oral administration, a suspension of lovastatin was prepared in 0.5% methylcellulose and administered by gavage. For dermal administration, lovastatin was mixed initially with 100% DMSO and in subsequent experiments, with hydrophilic petrolatum and applied to the back of the animals after shaving (area of application=6.45 cm²). In later experiments, the dermal formulation was modified and a aqueous alkanolic gel with a carbomer-based formulation containing water, ethanol, Carbomer 940, cholesterol, BHA and TEA was used.

### Serum Biochemistry

Blood samples were obtained at the end of the five day treatment for determination of liver and muscle enzymes (alanine aminotransferase (ALT), aspartate aminotransferase (AST), alkaline phosphatase (AP), and lactic dehydrogenase (LDH) by radioimmunoassays (Esoterix, San Antonio, Tex.). Kinetic quantitative determination of creatine protein kinase (CPK) in serum was estimated using a kit from Stanbio Laboratory (Boerne, Tex.). The concentration of osteocalcin was measured using a sandwich ELISA assay supplied by from Biomedical Technologies Inc.

### Assessment of Effects of Statins on Bone

Three-month old virgin female Sprague Dawley rats were purchased from Harlan Laboratories, LTD (Indianapolis, Ind.). Experiments were performed using either intact, bilaterally ovariectomized (OVX) or sham-operated (SHAM) rats with treatment starting 5 days after surgery in the latter groups. Rats were weight-matched and divided into treatment groups (n=10). Compounds were administered by daily transdermal application for 5 days only or 5 days/week for 5 weeks when specified. Animals were pair-fed throughout the experimental period and weekly body weights determined and dosage adjusted accordingly. At the completion of the experiment, animals were anesthetized with a ketamine (10 mg/ml) at a dose of 100 mg/kg body weight and euthanized by cervical dislocation. The study protocol was approved by the Animal Care and Use Committee at the University of Texas Health Science Center, San Antonio, Tex.

Following sacrifice, both femurs and tibiae were removed, cleaned of soft tissue, fixed in 10% formalin for 48 hours, and then stored in 70% ETOH and prepared for histology. Histomorphometric analysis was performed using a semiautomated Osteomeasure System (Osteometrics, Inc., Atlanta, Ga.) and digitizing pad and by following standard histomorphometric techniques. Bone volume, trabecular number, thickness and separation, cell number and dynamic parameters were determined as described previously by Parfitt et al. [Parfitt A M (1988) Bone histomorphometry: standardization of nomenclature, symbols and units. Summary of proposed system. J Bone Miner Res 4:1-5.]. Bone formation rates (BFR) and mineral apposition rates (MAR) were measured in plastic-embedded sections following demeclocycline and calcein injections (15 and 20 mg/kg/body weight respectively) given intraperitoneally at 10 and 4 days before sacrifice. Values for MAR were corrected for obliquity of the plane of section in cancellous bone. Rats were evaluated with a mouse densitometer, Piximus (GE Medical Systems); bone mineral density (BMD), calculated by dividing bone mineral content (g) by the projected bone area (cm²), was assessed for the proximal third of the tibia at time 0 and at 5 weeks. Micro-computed tomography (μ-CT) analysis of the rat distal femur was kindly performed by Phil Salmon (Skyscan, Belgium). Bones were scanned using the Skyscan Model 1072 employing an x-ray tube voltage of 100 kV, and magnified to attain a pixel size of 10.13 μm. Data are expressed as the mean standard error (SEM). Statistical differences between groups were evaluated with one-way analysis of variance (ANOVA). When the analysis of variance performed over all groups was significantly different among the groups, statistical differences between two groups were subsequently analyzed using Tukey's multiple comparison test. P<0.05 were considered significant.

### Biomechanical Testing of Femurs

Three month old rats were dosed with vehicle or transdermal lovastatin, 1 mg/kg/day for 5 days. Four weeks after dosing, rats were euthanized and femurs removed and stored frozen. Samples were thawed to room temperature on the day of testing, and remaining soft tissue was removed. To obtain mechanical properties, the femurs were subjected to three point bending with an EnduraTEC mechanical testing system (Elf 3300, Bose Corporation, Minnetonka, Minn.). Each rat femur was horizontally positioned on the support rollers (which were 12 mm apart) such that the vertical, rounded indenter loaded the femur with the medial side in front and the anterior side down (i.e., bending occurred about the medial-lateral axis). The force-displacement curve was recorded as the indenter traveled at rate of 3 mm/min into femur midshaft. Structural properties were obtained directly from the load deformation curves.

### Results

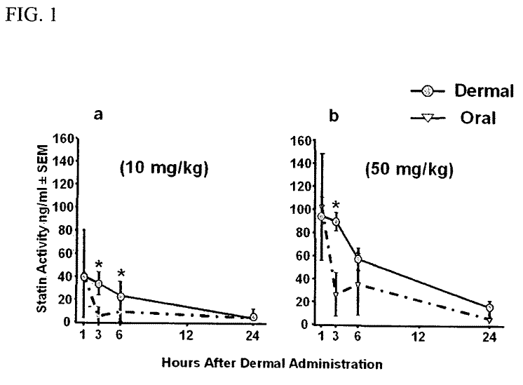

FIG. 1 shows plasma lovastatin levels of intact rats after a single dose of lovastatin administered orally or dermally at 1, 3, 6 and 24 hours. The level of the drug was determined as described in Material and Methods. Oral lovastatin was administered by gavage in 0.5% methylcelullose. For comparison, lovastatin was given dermally with application to the back of rats after shaving, using 100% DMSO as vehicle. Two different doses of lovastatin were administered as shown in panels a and b. Dermal application of lovastatin led to plasma concentrations of lovastatin which were greater, less variable and more prolonged than when the drug was given orally. Similar results were obtained with dermal application of lovastatin when hydrophilic petrolatum was substituted for DMSO as vehicle (data not shown). To determine the bone effects of lovastatin when applied dermally, experiments were conducted in three-month intact rats and ovx/sham rats. Lovastatin was mixed with hydrophilic petrolatum and applied to the back of the animals after shaving at a dose of 1 and 5 mg/kg/day for the first 5 days. The control group received hydrophilic petrolatum only. At the end of the five day treatment, serum was obtained to measure liver and muscle enzymes (ALT, AST, AP, LDH and CPK). No changes among lovastatin and vehicle-treated groups were observed (Table 1 below).

TABLE 1

| | | Lovastatin | Lovastatin |
|---|---|---|---|
| | Vehicle | 1 mg/kg/day | 5 mg/kg/day |
| AST (μ/L) | 114 ± 7 | 110 ± 4 | 128 ± 7 |
| ALT (μ/L) | 60 ± 2 | 55 ± 3 | 57 ± 2 |
| AP (μ/L) | 144 ± 7 | 123 ± 6 | 115 ± 5 |
| LDH (μ/L) | 486 ± 112 | 349 ± 47 | 528 ± 67 |
| CPK (μ/L) | 497 ± 60 | 441 ± 61 | 578 ± 45 |

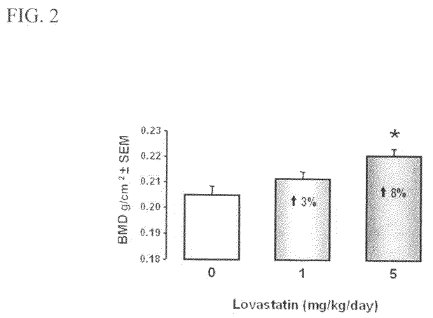

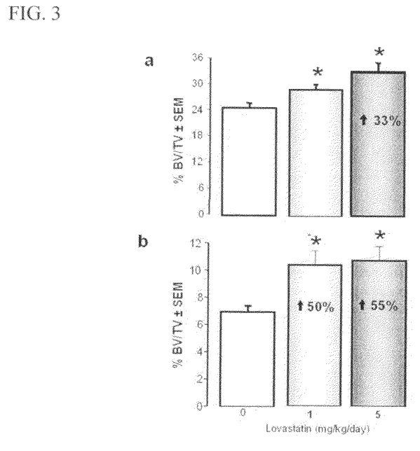

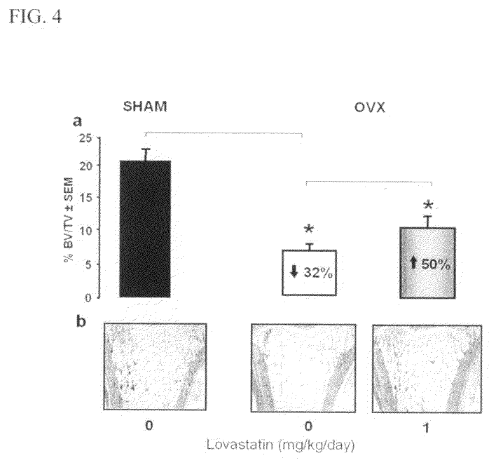

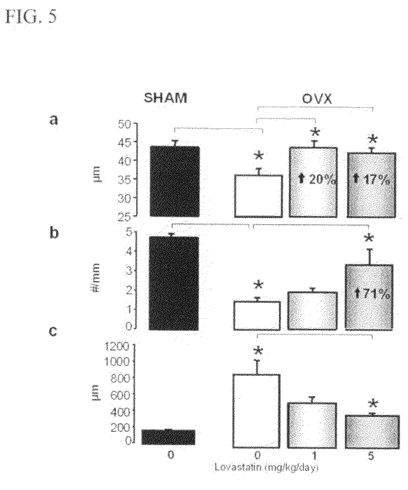

All animals were sacrificed four weeks after the treatment was discontinued and bones collected for quantitative bone histomorphometry in decalcified and non-decalcified sections as described in Materials and Methods. Weekly administration of dermal lovastatin in intact rats led to an increase of 8% in BMD (p<0.05) over the vehicle-treated controls (FIG. 2). Bone histomorphometric results are shown in FIG. 3. Bone volume in the proximal tibial metaphysis significantly increased when intact rats were treated with 1 and 5 mg/kg/day for 5 days only (17 and 33% respectively) as illustrated in FIG. 3a. Treatment of OVX rats with dermal lovastatin for 5 days increased bone volume by >50% compared to vehicle-treated OVX rats, even at the lowest dose (FIG. 3b). As shown in FIG. 4, five weeks after OVX, cancellous bone mass was significantly reduced (32%) in the proximal tibiae of vehicle-treated OVX rats relative to vehicle-treated SHAM controls as expected. When OVX rats were treated with dermal lovastatin (1 mg/kg/day) there was a 50% increase in bone volume compared to OVX rats treated with vehicle. Ovariectomy resulted in a decrease (compared to SHAM controls) of the structural indices of trabecular bone architecture as evidenced by significant changes in trabecular thickness, trabecular number and trabecular separation. Treatment of OVX animals with dermal lovastatin partly prevented these changes (FIG. 5).

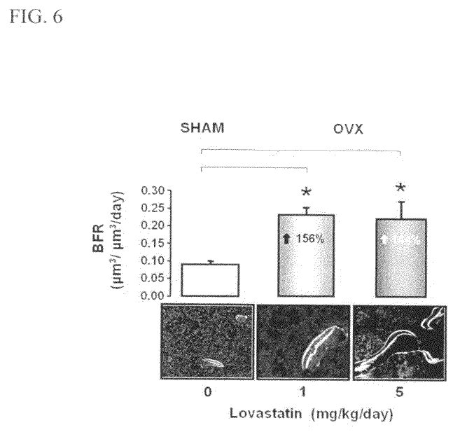

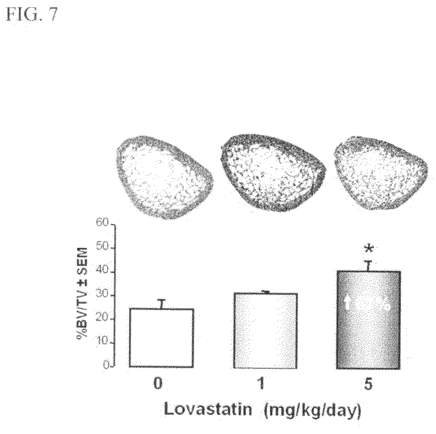

The increase in the volume of trabecular bone after dermal administration of lovastatin was accompanied by a significant increase in the bone formation rates (BFR) even in OVX rats as demonstrated in FIG. 6. The increase in BFR was mainly due to a substantial increase inactive mineralizing surfaces with mineral apposition rates slightly augmented. Bone formation rates were also significantly increased in intact rats: 166% at 5 mg/kg/day, data not shown). Trabecular architecture measured by μCT showed higher cancellous bone volume in the distal femoral metaphyses of lovastatin-treated intact rats versus controls (FIG. 7). This increase in bone volume was accompanied with an increase in trabecular thickness and number, and reduced trabecular spacing. Collectively, these data suggest a substantial anabolic effect of dermal lovastatin in this animal model.

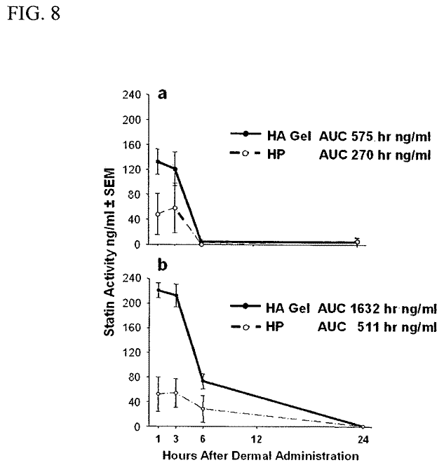

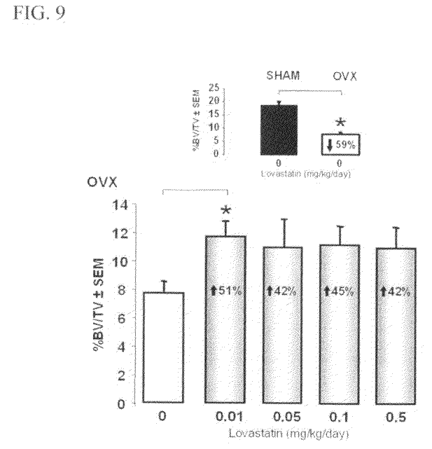

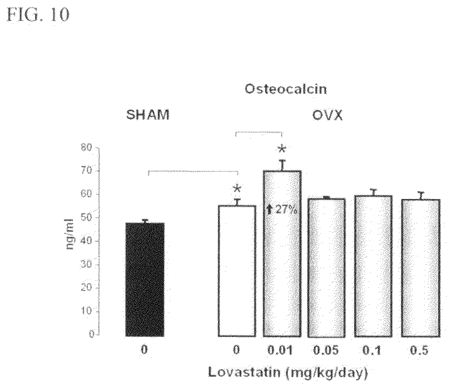

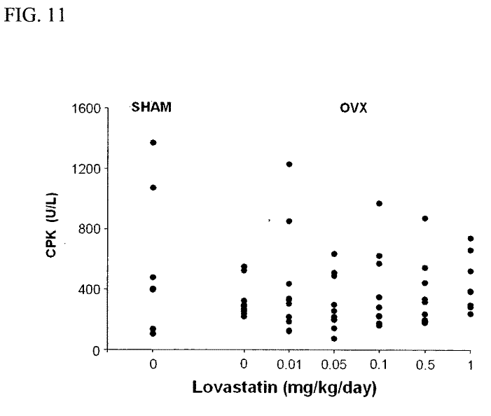

In order to improve the quality and characteristics of the dermal formulation for lovastatin, an aqueous alkanolic gel, based on carbomer 940 was developed and a biodistribution study was performed to compare this gel with hydrophilic petrolatum. Plasma drug levels at 1, 3, 6 and 24 hours after a single dose of dermal treatment with lovastatin in either hydrophilic petrolatum or aqueous alkanolic gel, were assessed by inhibition of the membrane bound HMG-CoA reductase assay as described earlier. Results are shown in FIG. 8. This gel formulation increased the dermal absorption of lovastatin with higher plasma levels than those obtained with hydrophilic petrolatum. Peak plasma levels were achieved within 3 hours using hydrophilic petrolatum and within the first hour with the aqueous alkanolic gel. The area-under-the-plasma-concentration curve (AUC0-24 h) for the aqueous alkanolic gel was more than double that of the petrolatum formulation at both doses tested. Since the aqueous alkanolic gel seemed to improve the bioavailability of lovastatin, a systemic experiment in sham/ovx rats was conducted using this gel as vehicle to determine if the efficacy of the drug in bone could be improved. When applied dermally in the aqueous alkanolic gel, lovastatin increased bone volume at all the doses tested (0.01 to 0.5 mg/kg/day), being significant at 0.01 mg/kg/day as assessed by bone histomorphometry (FIG. 9). There was also a significant increase in trabecular number and significant decrease in trabecular separation at the lowest dose tested (data not shown). At day 26, serum was collected for osteocalcin determination. As shown in FIG. 10, there was a significant increase in osteocalcin levels at the lower dose tested (0.01 mg/kg/day) No significant changes were detected in liver and muscle skeletal tissue enzymes (AST, ALT, AP, LDH and CPK) at the end of treatment. Results of CPK determinations are shown in FIG. 11.

To further evaluate the effects of transdermal lovastatin on bone, the biomechanical properties of intact femurs was evaluated after a 5 day treatment with lovastatin using the improved formulation. The biomechanical properties were determined using three-point bending as described in material and methods. Biomechanical data are presented in Table 2 below.

TABLE 2

| | | Bending | Stiff- | Modulus of |
|---|---|---|---|---|
| | Maximum | strength | ness | elasticity |
| | force (N) | (MPa) | (N/mm) | (MPa) |
| Vehicle | 132.3 ± 4.1 | 139.1 ± 3.2 | 456.0 ± 69.4 | 3926.5 ± 590.8 |
| Lovastatin | 141.7 ± 3.4 | 165.2 ± 3 | 561.3 ± 29.5 | 6379.9 ± 455.1 |
| (1 mg/ | | | | |
| kg/day) | | | | |

There was a significant increase in the bending strength of femurs of rats treated with dermal lovastatin (19% increase vs. Control) which indicates the treated rats had bones with higher strength that non-treated groups, therefore they were able to withstand higher force. Although non-significant, there was a trend for lovastatin-induced changes in all the biomechanical parameters obtained.

The results of this study show that transdermally administered lovastatin leads to plasma concentrations of HMG-CoA reductase inhibitor activity that are higher, maintained longer and less variable than those following oral administration (FIGS. 1 and 8). Moreover, the data also suggest that bone formation rates are markedly increased after only 5 days of exposure to transdermal lovastatin using doses of 0.01 mg/kg body weight. It is important to note that this dose is approximately 1/1000 of the dose required to produce a biological effect on bone formation when the drug is administered orally [Mundy G R, Garrett I R, Harris S E, Chan J, Chen D, Rossini G, Boyce B F, Zhao M, Gutierrez G (1999) Stimulation of bone formation in vitro and in rodents by statins. Science 286:1946-1949.]. These rates remained more than 150% greater than those of control rats after 30 days [Parfitt A M (1988) Bone histomorphometry: standardization of nomenclature, symbols and units. Summary of proposed system. J Bone Miner Res 4:1-5]. The increases in bone formation rates are also associated with substantial increases in trabecular bone volume when measured either by bone mineral density measurements or by quantitative histomorphometry. Transdermal lovastatin also increased cancellous bone connectivity, as assessed by trabecular thickness, number and separation, bone marrow star volume, fractal dimension, trabecular bone pattern factor, and structural analysis. Several of these effects exhibit flat dose-response curves (FIGS. 3 and 9). This behavior may be the result of a triggering phenomenon wherein even very small doses are sufficient to initiate a cascade of events that result in bone formation (see below). Alternatively, uptake to the site of action may be saturated at low drug concentrations. Whatever the mechanism, flat concentration-effects have been reported for many drugs (Reves J G, Fragen R J, Vinik H R, Greenblatt D J (1985) Midazolam: Pharmacology and uses. Anesthesiology 62: 310-24., Love J N (1994) Beta-blocker toxicity: A clinical diagnosis. Am J Emerg Med 12: 356-7.) including benzodiazepines (i.e. duration of apnea) and beta-blockers (i.e. intensity of hypotensive effect). Some of the statins have been shown to enhance bone formation in vitro and in vivo in ovariectomized (OVX) and in intact rats [Love J N (1994) Beta-blocker toxicity: A clinical diagnosis. Am J Emerg Med 12: 356-7., Frans J, Maritz Maria M, Conradie Philippa A, Hulley Razeen Gopal, Stephen Hough (2001) Effect of statins on bone mineral density and bone histomorphometry in rodents. Arterioscler, Thromb Vasc Biol. 21:1636., Oxlund H, Dalstra M, Andreassen T T (2001) Statin given perorally to adult 16 rats increases cancellous bone mass and compressive strength. Calcif Tissue Int 69:299-304., Oxlund H, Andreassen T T (2004) Simvastatin treatment partially prevents ovariectomy-induced bone loss while increasing cortical bone formation. Bone 34:609-18.]. However, the doses that are required for bone-related in vivo activity in rodents are many times greater than those used for cholesterol-lowering, if extrapolated to humans on a mg/kg basis (10 mg/kg vs. 0.1 mg/kg). This indicates that the dose required for oral administration of statins for the successful treatment and/or prevention of osteoporosis would be too high and be associated with unacceptable toxicity. In fact, when statin was extracted from bone and measured by the HMG-CoA reductase inhibition assay, extremely low statin levels were detected in the skeleton even with excessively high oral dosing (50 mg/kg/day, unpublished data). Improving peripheral distribution by using transdermal administration resulted in higher plasma statin levels and enhanced bone anabolic effects. These effects were achieved at significantly lower doses of the agent administered and for five days only.

One major concern of transdermal application of lovastatin was the possibility of the occurrence of myotoxicity at the doses required to stimulate bone formation. However, myotoxicity was not observed using doses up to 50 mg/kg/day as assessed by CPK measurements and morphologic examination of skeletal muscles (data not shown). The 50 mg/kg/day dosage level represents a 5000-fold increase from the experimental dosage level of 0.01 mg/kg which was found effective in stimulating bone formation. The mechanism responsible for myotoxicity following oral administration remains unknown and will require further investigation. The present results show myotoxicity does not occur with transdermal administration at the doses used to stimulate bone formation.

Statins are very safe drugs but have been associated with two rare but catastrophic toxic effects, specifically, hepatic necrosis and rhabdomyolysis with acute renal failure. Following oral administration, much of the absorbed drug is partitioned into the liver before reaching the systemic circulation (via the hepatic vein/vena cava). The liver therefore receives a much greater initial exposure to the orally administered drug than it does following transdermal or parenteral administration. Furthermore, preliminary results suggested that the total transdermal dose of lovastatin that produced a positive effect on bone would be much lower than the oral dose needed to produce the same effect. Since available evidence suggests both serious and minor statin toxicities (e.g., elevated liver enzymes) are dose dependent, transdermal delivery of this drug should provide a mechanism to minimize hepatotoxicity and myotoxicity while still achieving beneficial results. It has also been shown that cytochrome P450 3A enzymes are involved in the formation of most of the pharmacologically inactive metabolites present in human bile after oral administration of lovastatin [Wang R W, Kari P H, Lu A Y H, Thomas P E, Guengerich F P and Vyas K P (1991) Biotransformation of lovastatin: IV. Identification of cytochrome P450 3A proteins as the major enzymes responsible for oxidative metabolism of lovastatin in rat and human liver microsomes. Arch Biochem Biophys 290: 355-361]. Only metabolites of the drug are detected in the bile with no evidence of lovastatin or its open-ring form [Wang R W, Kari P H, Lu A Y H, Thomas P E, Guengerich F P and Vyas K P (1991) Biotransformation of lovastatin: IV. Identification of cytochrome P450 3A proteins as the major enzymes responsible for oxidative metabolism of lovastatin in rat and human liver microsomes. Arch Biochem Biophys 290: 355-361.]. The two major products of lovastatin after metabolism by the liver are 6′-hydroxy and 6′-exomethylene lovastatin. 6′-Hydroxylovastatin formation in the liver is inhibited by the specific CYP3A inhibitors cyclosporine, ketoconazole and troleandomycin and potentially many other substrates for cytochrome P450 3A [Jacobsen W, Kirchner G, Hallensleben K, Mancinelli L, Deters M, Hackbarth I, Benet L Z, Sewing K F, Christians U (1999) Comparison of cytochrome P-450-dependent metabolism and drug interactions of the 3-hydroxy-3-methylglutaryl-CoA reductase inhibitors lovastatin and pravastatin in the liver. Drug Metab Dispos 27:173-9.]. These interactions usually involve a substantial decrease in the extent of first pass metabolism (liver and/or gut wall) and some decrease in total body clearance. Transdermal administration by definition eliminates the first pass component of these interactions. Furthermore, except for the possibility of skin irritation or toxicity to tissues directly under the skin at the site of application, it is difficult to postulate how transdermal application of identical doses could be as toxic as orally administered drug.

Thus, efficacy was observed at transdermal doses which are a small fraction of the dose required for oral activity and pharmacologic theory and available clinical observations [Chen H S, Gross J F (1980) Intra-arterial infusion of anticancer drugs: theoretic aspects of drug delivery and review of responses. Cancer Treat Rep 64:31-40., Bland L B, Garzotto M, DeLoughery T G, Ryan C W, Schuff K G, Wersinger E M, Lemmon D, Beer T M (2005) Phase II study of transdermal estradiol in androgen-independent prostate carcinoma. Cancer 103:717-23., Utian W H (1987) Transdermal estradiol overall safety profile. Am J Obstet Gynecol 156:1335-8., Wemme H, Pohlenz J, Schonberger W (1995) Effect of oestrogen/gestagen replacement therapy on liver enzymes in patients with Ullrich-Tumer syndrome. Eur J Pediatr 154:807-10.] suggest greater intrinsic safety at least with regard to hepatic toxicity.

### Experiment 1—Systemic Administration

The study consisted of 5 groups (n = 12)

| Group 1. | Vehicle | | |
|---|---|---|---|
| Group 2. | Lovastatin PO | 10 | mg/kg/day |
| Group 3. | Lovastatin PO | 25 | mg/kg/day |
| Group 4. | Lovastatin TD | 1 | mg/kg/day |
| Group 5. | Lovastatin TD | 2.5 | mg/kg/day |

(PO—oral gavage; TD—transdermal)

### Experiment 2—Systemic Administration

The study consisted of 5 groups (n = 12)

| Group 1. | Vehicle | | |
|---|---|---|---|
| Group 2. | Lovastatin TD | 0.1 | mg/kg/day |
| Group 3. | Lovastatin TD | 1 | mg/kg/day |
| Group 4. | Lovastatin TD | 5 | mg/kg/day |
| Group 5. | Lovastatin PO | 5 | mg/kg/day |

### Radiographs

### Experiment 1-1—Systemically Delivered Lovastatin

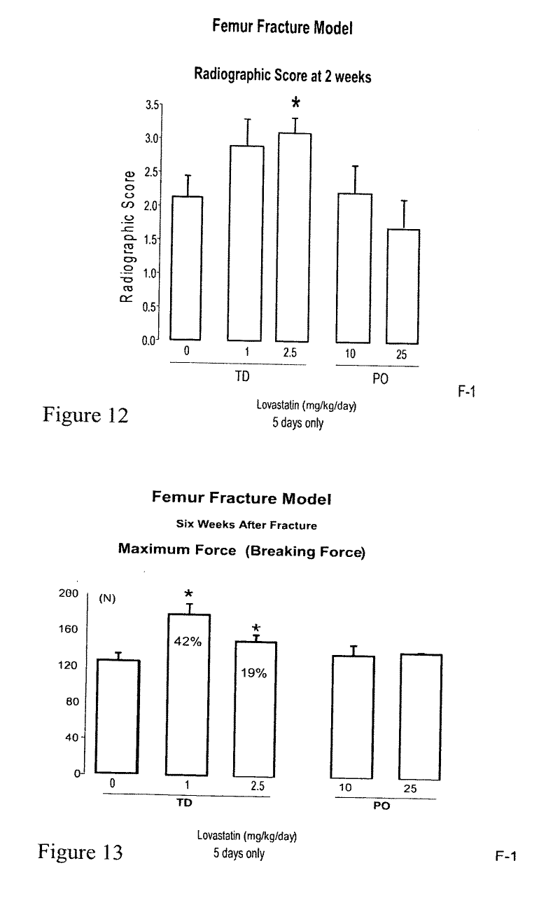

Radiographs at two weeks were assessed blindly by two investigators using a scoring scale devised by one of them, based on rebridgement of the cortices and acceleration of healing (FIG. 12). The scoring was based on blinded observer assessment of rebridging of the cortices based on the following scale:

| Score | Interpretation |
|---|---|
| 0 | no rebridgement |
| + | rebridgement of one cortex or evidence of callus |
| ++ | rebridgement of two cortices |
| +++ | rebridgement of three cortices |
| ++++ | rebridgement of all four cortices |
| +++++ | full rebridgement and remodeling of the defect |

In summary, transdermal lovastatin caused a striking effect at both doses at 2 weeks; oral lovastatin treatment showed no difference from vehicle-treated controls. Radiological evaluation of rats receiving transdermal lovastatin showed enhanced fracture repair so that there was complete healing by week 6 (FIG. 12). However there was no difference between 1 and 2.5 mg/day. Oral treatment at high doses 10 and 25 mg/kg showed no difference between the treated and the controls at six weeks. These results suggest that at high doses orally there was no enhancement of bone fracture repair and at the lower transdermal doses there was enhancement of fracture repair but a maximum was achieved when doses at 2.5 mg/kg/day for 5 days. This indicates the maximum dose required for transdermal delivery of lovastatin is 2.5 mg/kg/day and that 10 mg/kg/day oral dosing is ineffective. It appears for transdermal dosing the most effective dose is 0.1 mg/kg/day for 5 days.

### Experiment 1—Systemic Delivered Lovastatin

At 6 weeks, femurs of rats treated with transdermal lovastatin were significantly stronger than the controls. The force required to break the bone was 42% greater than vehicle treated controls. However it is clear that the 5 day transdermal dose of 2.5 mg/kg resulted in a lower maximum force than the 1 mg/kg/day dose to break the bones. These results indicate that higher does are not necessarily better and appear to be deterimental. Oral lovastatin had no effect at 10 and 25 mg/kg/day indicating oral doses are not effective even at these high doses. See FIG. 13.

### Experiment 2—Systemic Delivered Lovastatin

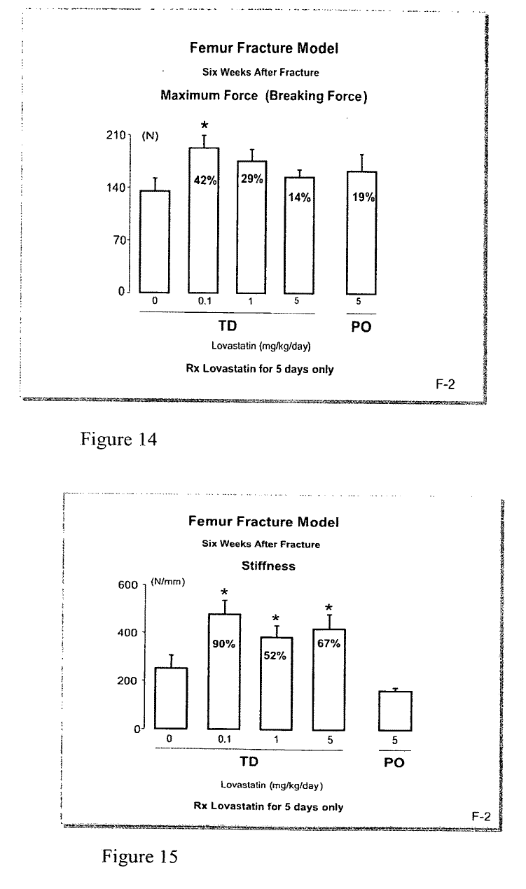

At 6 weeks, femurs of rats treated with transdermal lovastatin were significantly stronger than the controls. The force required to break the bone was 42% greater than vehicle-treated controls when using 0.1 mg/kg/day of TD lovastatin. This data confirms the results seen with radiographs for this experiment—doses higher than 0.1 mg/kg/day resulted in a reduced maximum force to rebreak these bones. Oral lovastatin had no effect at 5 mg/kg/day. See FIG. 14.

While oral lovastatin showed an increase in stiffness in the previous experiment where higher doses were tested, there was no effect in this experiment at 5 mg/kg/day. This data confirms the results seen with radiographs and maximum force for this experiment—doses higher than 0.1 mg/kg/day resulted in a reduced maximum force to re-break these bones. See FIG. 15.

### Plasma Lovastatin Levels

### Experiment 2—Systemically Delivered Lovastatin

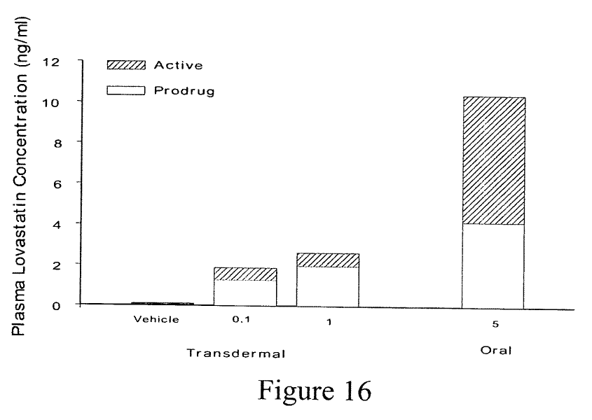

Plasma was taken from the rats 3 hrs after the last dose and the lovastatin was measured by mass spectroscopy. FIG. 16—At 3 hrs after the last dose oral dosing at 5 mg/kg/day showed up as 10 ng/ml whereas the most effective transdermal doses 0.1 and 1 mg/kg/day showed plasma lovastatin levels of only 2-3 ng/ml. Effective plasma levels from transdermal administration is on the order of 2-3 ng/ml.

### Nanoparticles

### Nanoparticle Study 1

### Preparation of Nanoparticles:

Mix the following components:
1 ml of 100 mg/ml poly(DL-lactide) DLPLA η 0.26-0.54 dissolved in acetone from stock solution from (Durect Corporation Cat# 100D040A)
0.4 ml of 50 mg/ml Lovastatin in acetone
8.6 ml acetone (Fisher Cat#A949-1)
Ratio PLA-Lovastatin 1:5. 10 ml acetone final volume
The final 10 ml solution is dialyzed in 10 KD cassette Cat # 66807against 3 liter of water, changed dialysis every 3 hours at room temperature five times with a stir bar mixing set at 5 in the dial. Take 200 μl of the suspension and measure lovastatin levels by HPLC, and another 200 μl to determine the total weight. Use this information to determine the total lovastatin loading. Collect the nanoparticles with centrifugation at 10,000 rpm and lyophilize for long term storage.
The rats employed are 3-month old Sprague-Dawley virgin female rats of 8-10 weeks age at initiation, 200-250 g. Animals are purchased from Harlan laboratories and housed at the University of Texas Health Science Center at San Antonio, laboratory animal facility.
Microsphere Preparation with Surfactant.

Five grams of the polymer 85/15 DLPLGA (DL-polylactic-glycolic acid, Durect) were dissolved in 25 ml of methylene chloride to give a 1:5 weight/volume ratio. A 1% solution of poly (vinyl alcohol) (PVA mw=25 kdal, 88% mole hydrolyzed (Sigma, Inc.)) was used as a surfactant. The DLPLGA solution was added dropwise to 1% PVA solution with stirring (300 rpm) overnight. This allowed the complete evaporation of the solvent. The microspheres were isolated by vacuum filtration, washed with deionized water, air dried for 2 h and then vacuum dried overnight. Microspheres were kept in a desiccator until further use. The free flowing microspheres were then sieved into the following size ranges using micron size sieves: 150 μm, 250 μm, 500 μm and 1 mm. For agglomeration, one can use one of the following methods:

1. By packing the beads into a defined shape—plastic or metal tubes are used of varying diameter and ethanol is applied to the packed beads by poring though the beads. This has the effect of slightly melting the beads allowing them to fuse together, followed by repeated washing.
2. An alternative method was to pack the beads and use heat at 50° C. for 1 hr to slightly melt the beads allowing them to fuse together.

### Experimental Methodology

A study is performed to demonstrate the effect of controlled-release local lovastatin, exemplified by evaluating the enhancement of fracture repair in rats. The purpose of this study is to demonstrate that controlled-released lovastatin administered locally by a single injection can enhance callus formation and fracture repair that leads to accelerated restoration of mechanical stability. The test material is lovastatin in nanoparticles prepared as described above. The preparation is of at least 99% purity and is a white to off-white powder. The test articles are nanoparticles with and without lovastatin. The particles in a vehicle are injected at the fracture site in a volume of 50 μl to provide 10.5, 52.5, 75.7 or 378 μg total lovastatin. The lovastatin levels are determined by HPLC and the release curved is followed throughout the experiment.

In accordance with the study, the clinical focus involves creating uniform and reproducible fracture defects utilizing a pinned closed transverse rat femoral model chosen because it has been well defined and fully characterized by mechanical and histologic methods. Advantages of this model include reproducibility, defect uniformity, and a rapid 5 weeks to clinical union healing phase. The properties of the bioactive coating are investigated in preliminary studies in vitro and in vivo using the explanted calvarial culture and the local calvarial injection model including drug-release kinetics, degradation and stability. The aims of the study are: (1) to evaluate the effect of controlled-released locally administered lovastatin on callus formation, progression and fracture healing using X-ray analysis of fracture healing. At the end of the experiment, the fractured limb will be excised and X-rayed after removal of stabilizing pins. These X-rays will be assessed for evidence of healing of the fracture. They will be scored by 3 independent observers for healing of the fracture; (2) to evaluate the effect of controlled released lovastatin on biomechanical parameters by three-point bending and micro computer tomography (uCT); and (3) to evaluate by uCT bone microarchitecture at callus site and bone healing.

The experimental design is to use the rat long bone model in light of the application of these compounds in the orthopedic field. Three-month old female Sprague-Dawley rats are used; all animals undergo pinning of the femur followed by closed fracture of the mid diaphysis to create a transverse fracture. Lovastatin nanoparticles are injected at the site of the fracture (assessed by PIXI and x-rays). Animals are maintained for 3 weeks after surgery and euthanized at the end of the respective study period.

The female rats are treated pre-operatively with 0.25 cc Pen B+6 to prevent post-op infections. They are anesthetized with an injectable anesthetic (dormitor and ketamine) and the medial aspect of the femur is clipped and prepared for aseptic surgery. A hole is created in the medial tuberosity and a 20 g needle is used to ream the medullary cavity to its distal extent. A coated probe is placed down the medullary canal and seated in the distal femur, the wire cut flush with the bone and the skin repositioned to cover the pin. The rat is placed in a fracture device where the femur rests against the outer two supports. A 500 gm weight is dropped 40 cm to drive the anvil and fracture the bone. The leg is X-rayed to examine the fracture and fixation. Only animals with transverse fractures are accepted in the study. Additional radiographs are obtained as scheduled. Once the fracture is confirmed, nanoparticles are injected in the fracture site (50 μl PBS). The release rate for the lovastatin is about 2%/day.

Unrestricted activity is allowed after recovery from anesthesia. The animals are sacrificed six weeks after fracture surgery and the femora collected. The intramedullary wires are extracted and the femora dissected free of soft tissues.

For comparing data between the experimental groups, the paired student t-test is used. For multiple comparisons between more than two groups of data, such as different concentrations of factor treatment, one-way analysis of variance (ANOVA) will be used followed by Dunnett's test. Significant differences will be considered when a p<0.05 is found.

Lovastatin released from the nanobeads per day based on the amount of nanobeads applied is shown in the graph in FIG. 18 showing the radiographic score with the different amounts of lovastatin. Maximum radiographic score is achieved at a release of 1.5 ug/day. The lowest lovastatin amount tested that produced a significant increase in radiographic score was equivalent to 0.2 ug/day or 200 ng/day release per day.

The systemic exposure is:

0.2 ug dose=0.0008 mg/kg/day
1.0 ug dose=0.004 mg/kg/day
1.5 ug dose=0.006 mg/kg/day
7.5 ug dose=0.03 mg/kg/day
The assumptions for the systemic exposure are that: local release in vivo was the same as release in vitro 1-2%; constant release over 2 weeks; nanobeads injected directly into fracture; lovastatin stable in nanobeads over entire experiment; and the rat weight was 250 g. Doses were based on the above rat data.

The scaling by fracture surface area was calculated as follows using the following assumptions: fracture is cross sectional area of femur−rats femur diameter=5 mm (area=20 mm²), human femur diameter=30 mm (area=700 mm²), human weight 70 kg. The lovastatin dose by cross sectional (fracture) area=0.00001-0.000375 mg/mm²/day. The total human dose of lovastatin per day would be =0.007-0.26 mg per day for a 700 mm² fracture area; treatment period=10 days; total exposure for 10 days=0.07-2.6 mg. Based on a 70 kg body weight of a human, the systemic exposure of statin per day would equal 0.0001-0.0037 mg/kg/day.

### Experiment A—Local Administration

The study consisted of 5 groups (n = 12)

| Group 1. | Vehicle PBS | | |
|---|---|---|---|
| Group 2. | Vehicle - nanobeads | 0 | ug/day |
| Group 3. | Lovastatin nanobeads | 0.2 | ug/day |
| Group 4. | Lovastatin nanobeads | 1.0 | ug/day |
| Group 5. | Lovastatin nanobeads | 1.5 | ug/day |
| Group 6. | Lovastatin nanobeads | 7.5 | ug/day |

### Results

Midshaft transverse fractures were induced in all animals. Fractures were tolerated and remained immobilized without surgical complications. Animals were freely mobile after recovery from anesthesia. Callus formation was observed on radiographic examination by 2 weeks in all animals.

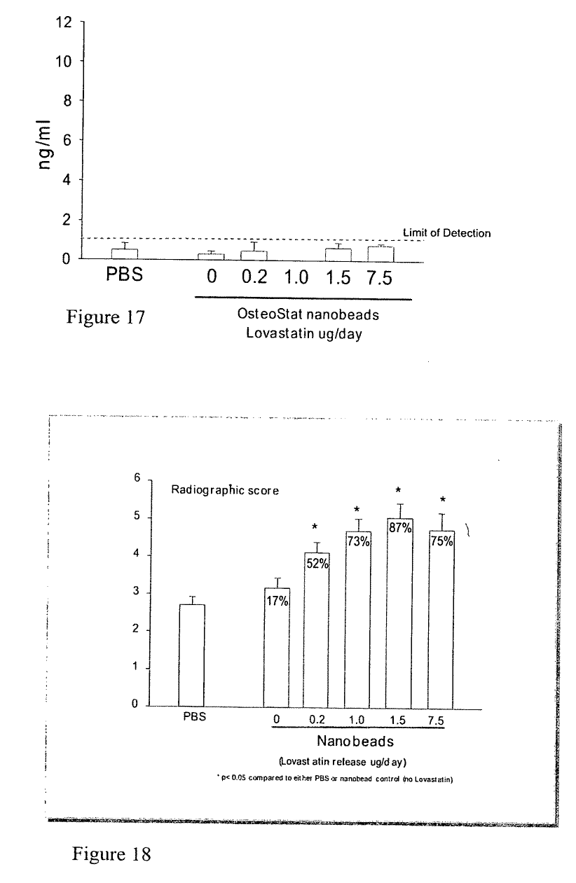

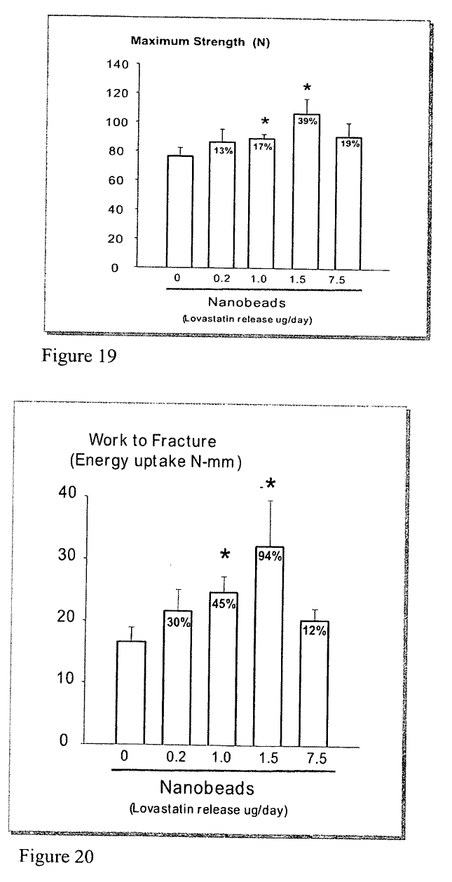

Parameters measured 1. X-ray assessments at 2 weeks and biomechanical testing. The results are shown in FIGS. 19 and 20. Blood was taken for plasma lovastatin assessments. See FIG. 17.

Lovastatin delivered locally by a single injection of nanobeads containing lovastatin markedly improved the radiographic scoring at 2 weeks in a dose dependent manner with a maximum effect occurring at 1.5 ug of lovastatin released per day. Above this dose there did not appear to be any further enhancement of fracture repair.

### Experiment A —Locally Delivered Lovastatin

Radiographs at two weeks were assessed blindly by two investigators using a scoring scale from 0-7 based (see below), based on rebridgement of the cortices and acceleration of healing. The scoring was based on blinded observer assessment of rebridging of the cortices based on the following scale:

| Fracture | Score | |
|---|---|---|
| [symbol] | 0 | No bridging, no callus formation |
| [symbol] | 1 | No Bridging, initiation of a small amount callus |
| [symbol] | 2 | No bridging, obvious initial callus formation near fracture |
| [symbol] | 3 | |
| [symbol] | 4 | No bridging marked callus formation near and around fracture |
| [symbol] | 5 | No bridging, marked callus formation near and around fracture site. |
| [symbol] | 6 | Rebridging of at least one of the cortices, marked callus formation near and |
| | | around fracture site |
| [symbol] | 7 | Rebridging of both cortices, and/or some resolution of the callus |

Clear rebridging of both cortices and resolution of the callus

### Experiment A—Locally Delivered Lovastatin

Plasma was taken from the rats 3 hrs after the last dose and the lovastatin was measured by mass spectroscopy. FIG. 17—At the end of the experiment local administration of plasma lovastatin was undetectable in any of the groups dosed with lovastatin indicating this is a local effect.

### Nanoparticle Study 2

### Experimental Methodology

Male, Swiss ICR mice will be used (25-28 gm). Animals will be fed normal chow and allowed free access to water and housed in appropriate cages. Unrestricted activity will be allowed during the entire experiment. Before injection head will be shaved and thickness of the calvaria (left and right) will be recorded using a PalmScan AP2000. All injections will be performed on the right side of the calvaria. The left side will be used as controls.

### Preparation of Drugs

The solid lovastatin was weighed and broken into small particles using a mortar and pestle. A solution containing 25% PG-400 and 75% PBS was added to the mortar and the dispersion mixed well, followed by transfer with a pipette to a microcentrifuge tube. The dispersion is continuously agitated to obtain a homogeneous dispersion for injection.

The following table indicates the three compositions for testing and their properties.

| | | | | | |
|---|---|---|---|---|---|
| B | 25/75 | 0.0025 | 0.005 | 0.025 | g/mL |
| | | 2.5 | 5 | 25 | μg/μL |

| | | | | Mass in | Microvial | |
|---|---|---|---|---|---|---|
| | Mass | Volume | | injection | concentration | Percent |
| | (g) | (mL) | Area | (μg) | (μg/μL) | dissolved |
| B1 | 0.0024 | 0.96 | 4827 | 0.163 | 0.065 | 2.583 |
| B2 | 0.0047 | 0.94 | 4954 | 0.167 | 0.067 | 1.339 |
| B3 | 0.0302 | 1.208 | 6960 | 0.235 | 0.094 | 0.376 |

### Experimental Design

Swiss ICR white male mice 4-5 weeks old are used.

The animals are divided into the following treatment groups. Injection volume: 50 ul.

Sacrifice after

Vehicle groups 3-8: 25% PG400-75% PBS.

| | |
|---|---|
| Gp1. 1-5 - Vehicle control 25%/75% PG400/PBS. | 3 weeks. |
| Gp2. 6-10 - Vehicle control 25%/75% PG400/PBS. | 7 weeks. |
| Gp3. 11-15 - Lovastatin 125 ug/50 ul once. | 3 weeks. |
| Gp4. 16-20 - Lovastatin 125 ug/50 ul once. | 7 weeks. |
| Gp5. 21-25- Lovastatin 250 ug/50 ul once. | 3 weeks. |
| Gp6. 26-30- Lovastatin 250 ug/50 ul once. | 7 weeks. |
| Gp7. 31-35- Lovastatin 1250 ug/50 ul once. | 3 weeks. |
| Gp8. 36-40- Lovastatin 1250 ug/50 ul once. | 7 weeks. |

Vehicle groups 9-12: 0.1% BSA/PBS

| Gp9. 41-45 - Vehicle control 0.1% BSA/PBS | | | 3 weeks. |
|---|---|---|---|
| times/day × 3 d. | | | |
| Gp10. 46-50 - Vehicle control 0.1% BSA/PBS | | | 7 weeks. |
| 3 times/day × 3 d. | | | |
| Grp11 51-55 - aFGF | 104 ug/50 ul | 3 times/day × 3 d. | 3 weeks. |
| Grp12 56-60 - aFGF | 104 ug/50 ul | 3 times/day × 3 d. | 7 weeks. |

n=5/group.

### Standard Histological Measurements.

The total bone area in the right calvaria, bone width and osteoid surface are determined. Toxic effects are also checked.

### Statistical and Power Analysis

For comparing data between the experimental groups, the paired student t-test is used. For multiple comparisons between more than two groups of data, such as different concentrations of factor treatment, one-way analysis of variance (ANOVA) is used followed by Dunnett's test. Significant differences are considered when a p<0.05 is found.

Following the above procedure bone enhancement is obtained as is expected from the previous studies.

### CONCLUSIONS

Using a well established model of fracture repair in the rat, we have shown that transdermal lovastatin accelerates fracture healing. This was shown by both radiographic examination as well as biomechanical loading. The two fracture studies indicate an increase in both strength and stiffness in fractured bones when treated with transdermal lovastatin even at the lower dose of 0.1 mg/kg/day for 5 days only.

The most effective local dose in all assessments was 1.5 ug/day. The release profile of these nanobeads at best was estimated to be 2% per day. This would equate to a total release over 50 days essentially a continuous release delivery over the experimental period. Even with the 7.5 ug/day delivery (equivalent to 30 ug/kg/day) there were no detectable circulating levels of lovastatin suggesting strongly that the delivery of lovastatin nanobeads in improving fracture healing was a local and not a systemic effect.

1. Oral dosing at high doses of lovastatin did not enhance fracture repair
2. Systemic transdermal dosing of lovastatin did enhance fracture repair

The major therapeutic need in the field of osteoporosis is an agent that will increase bone formation and cause an anabolic effect on the skeleton with minimal side effects. Parathyroid hormone, fluoride and the peptide bone growth factors stimulate bone formation, but none are ideal in the clinical setting. Parathyroid hormone has now been approved by the FDA for treatment of osteoporosis [Arnaud, C D (2001) Two years of parathyroid hormone 1-34 and estrogen produce dramatic bone density increases in postmenopausal osteoporotic 17 women that dissipate only slightly during a third year of treatment with estrogen alone: Results from a placebo-controlled randomized trial. Bone 28: S77.], but it is a peptide that must be given by injection, not an ideal therapy for a chronic disease of the elderly. Fluoride is associated with impairment in mineralization of bone and bone fragility that results in bones still susceptible to fracture [Inkovaara J, et al. (1975) Prophylactic fluoride treatment and aged bones. Br Med J. 3: 73-74., Gerster J C, et al. (1983) Bilateral fractures of femoral neck in patients with moderate renal failure receiving fluoride for spinal osteoporosis. Br Med J 287(6394):723-5., Dambacher M A, et al. (1986) Long-term fluoride therapy of postmenopausal osteoporosis. Bone. 7: 199-205.]. The peptide growth factors also have growth effects on other tissues, which makes their administration for a chronic disease such as osteoporosis problematic. Moreover, these recombinant molecules must also be given by frequent injection.

Thus, there remains a great need for an efficacious therapy for osteoporosis that has acceptable toxicity and would not require administration via the parenteral route. The current preclinical data in rats suggests that transdermal lovastatin has the potential to fulfill these requirements.

As reported previously, statins enhance the expression of BMP-2 [Mundy G R, Garrett I R, Harris S E, Chan J, Chen D, Rossini G, Boyce B F, Zhao M, Gutierrez G (1999) Stimulation of bone formation in vitro and in rodents by statins. Science 286:1946-1949.]. BMPs are the most potent inducers and stimulators of osteoblast differentiation. They stimulate osteoprogenitors to differentiate into mature osteoblasts and also induce nonosteogenic cells to differentiate into osteoblast lineage cells [Wozney J M, Rosen V: (1998): Physiology and Pharmacology of Bone. Mundy J R, Martin T J Eds. Springer-Verlag, Chapter 20: 725-748.]. The present inventors have previously reported on the effect of statins in bone when administered orally [Mundy G R, Garrett I R, Harris S E, Chan J, Chen D, Rossini G, Boyce B F, Zhao M, Gutierrez G (1999) Stimulation of bone formation in vitro and in rodents by statins. Science 286:1946-1949.]. The present study shows the effects in bone of lovastatin when administered transdermally and with slow release particles. The extent of the effect observed is unprecedented, as graphically shown following transdermal administration. After only 5 days of administration, there was a profound effect on bone formation rates that was still apparent 5 weeks later. Although the cause for this long-lasting effect has not been investigated, it is most likely that triggering bone formation by enhancing the expression of BMP-2 leads to a number of secondary effects. These secondary effects include stimulation of cell proliferation and production of a number of other growth factors by the proliferating bone cells, including bone morphogenetic protein-4. As a consequence, once the bone formation process is initiated by the statin, it may well persist for some time. Indeed, this mechanism may also be responsible for the somewhat unusual dose-response data reported here.

Similar results to those observed with transdermal administration were observed with injection of slow release particles, as lovastatin in substantially pure form or as impregnated in a carrier.

### Cartilage Study

Experimental Summary: Determine the effectiveness of varying does of Lovastatin scaffolds on cartilage formation using murine calvarial cultures. Four day old calvarial explant cultures were incubated with media containing scaffold material that releases 0, 0.4 or 8 μg/day. The extent of new cartilage is then quantitated by imaging analysis.

Experimental Design Four day old Swiss white pups were employed as they are generally a healthy mouse strain. Calvaria from 4-day old Swiss white mouse pup mice were dissected out and cut in half. The excised hemi-calvariae were placed on metal grids (at the surface) in 1 ml BGJ media with Fitton-Jackson modification BGJ media (Sigma) containing 0.1% BSA with glutamine. The bones are incubated at 37° C. in a 5% humidified incubator for a period of 24 h and then transferred to wells containing 1 ml media with test compounds and further incubated under the above conditions for 72-96 h. The bones are then removed, fixed in 10% buffered formalin for 24 h, decalcified in 14% EDTA overnight, embedded in paraffin and 4 μm-thick sections cut and stained with H&E.

Dosing. Lovastatin scaffold material (LPGA polymer scaffold impregnated with 2.5 mg lovastatin, 5 mg pieces, estimated release is 0.4 μg/24 h) applied for the first 48 h and then removed. Calvaria are removed at day 7 and day 14. The media is changed every 3 days. Cartilage formation is assessed histologically.

| | | Dose | Time-Days | | |
|---|---|---|---|---|---|
| | | 24 h | Calvarial | | #Animals |
| Group | Treatment | Exposure | incubation | # Calvaria | (pups) |
| 1 | Control | Vehicle | 7 and 14 | 8 | 4 |
| 2 | BMP2 | 100 ng/ml | 7 and 14 | 8 | 4 |
| 3 | Lovastatin | 0.4 μg/24 h | 7 and 14 | 8 | 4 |
| 4 | Lovastatin | 0.8 μg/24 h | 7 and 14 | 8 | 4 |
| | | | | | 20 pups |

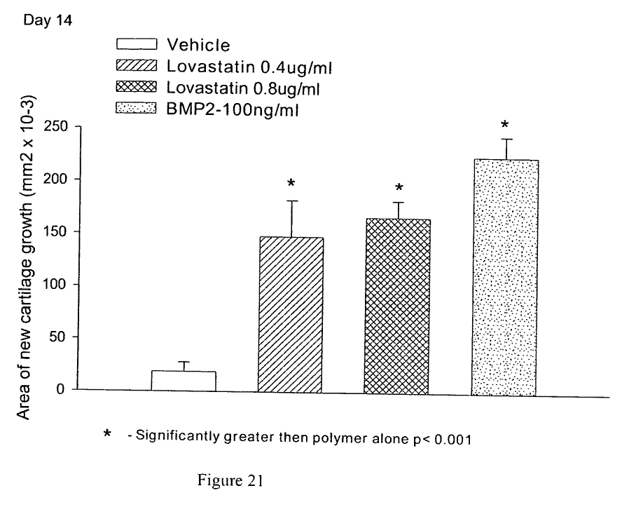

The results are shown in FIG. 21 as a bar graph. What is observed is that lovastatin stimulates bone formation in cultures of neonatal murine calvaria 7 days after exposure and cartilage formation 14 days after exposure. BMP stimulates bone formation in culture of neonatal murine calvaria 7 days after exposure. Lovastatin is shown to stimulate cartilage formation in a dose response fashion in cultures of neonatal murine calvaria.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method for enhancing mammalian skeletal framework tissue comprising:
administering to a mammalian host HMG-CoA reductase inhibitor with a biodistribution profile to provide a bioavailable dosage to said tissue for a time sufficient to enhance said skeletal framework, wherein the dosage provides enhancement to said tissue while minimizing bioavailability of said HMG-CoA reductase inhibitor to non-skeletal tissue and said time is selected to substantially minimize degradation of said enhancement.

2. A method according to claim 1, wherein said administering is using particles comprising said HMG-CoA reductase inhibitor.

3. A method according to claim 2, wherein said bioavailable dosage is in the range of about 0.1 to 5 μg/day for a rat and about 5 to 250 μg/day for a human and said duration is in the range of greater than one day and less than about 65 days.

4. A method according to claim 1, wherein said administering is using topical application.

5. A method according to claim 4, wherein said dosage is 0.01 to 10 mg/kg/day.

6. A method for enhancing bone and/or cartilage at a site of interest in a mammalian host, said method comprising:
administering at said site of interest slow release biocompatible particles of a size in the range of about 0.001-100 μm comprising HMG-CoA reductase inhibitor at a bioavailable dosage to provide a blood level of from about 0.5 to 5 ng/ml and for a time sufficient to enhance said skeletal framework, wherein the dosage is selected to provide enhancement while minimizing bioavailability of said HMG-CoA reductase inhibitor to non-skeletal tissue and said time is selected to substantially minimize degradation of said enhancement.

7. A method according to claim 6, wherein said HMG-CoA reductase inhibitor is a statin, said particles are nanoparticles of mean diameter in the range of about 0.1 to 100 nm and said time is greater than about 1 day and less than about 25 days.

8. A method according to claim 6, wherein said HMG-CoA reductase inhibitor is a statin and said particles are microparticles of mean diameter in the range of about 1 to 200 μm.

9. A method according to claim 6, wherein said wherein said HMG-CoA reductase inhibitor is in an amount of from 10 to 100% of said particles.

10. A method according to claim 6, wherein said HMG-CoA reductase inhibitor is admixed with a polymeric matrix.

11. A method for enhancing bone and/or cartilage at a site of interest in a mammalian host, said method comprising:
administering to said mammalian host by topical application at a biological surface HMG-CoA reductase inhibitor at a bioavailable dosage to provide an average blood level of from about 0.5 to 5 ng/ml during the course of treatment and for a time sufficient to enhance said skeletal framework, wherein the dosage is selected to provide enhancement while minimizing bioavailability of said HMG-CoA reductase inhibitor to non-skeletal tissue and said time is selected to substantially minimize degradation of said enhancement.

12. The method of claim 11, wherein said dosage in the range of about 0.1 to 5 mg/kg/day.

13. The method of claim 11, wherein application of an amount of the pharmaceutical composition onto said biological surface of said subject is capable of elevating a blood serum concentration of said HMG-CoA reductase inhibitor in said subject to 1-40 ng/ml within 1-2 hours.

14. The method of claim 11, wherein a surface area of said biological surface of said subject is 4-8 cm².

15. The method of claim 14, wherein said HMG-CoA reductase inhibitor is present in an amount between about 0.1 and about 10 mg/cm² of said biological surface.

16. The method of claim 15, wherein said biological surface is skin or mucosa.
